(19) United States
(12) Patent Application Publication
(10) Pub. No.: US 2020/0197533 A1
(43) Pub. Date: Jun. 25, 2020

(54) COMBINATION CANCER THERAPY USING CHIMERIC ANTIGEN RECEPTOR ENGINEERED NATURAL KILLER CELLS AS CHEMOTHERAPEUTIC DRUG CARRIERS

(71) Applicant: University of Southern California, Los Angeles, CA (US)

(72) Inventors: Pin Wang, Los Angeles, CA (US); Elizabeth Siegler, Los Angeles, CA (US); Xianhui Chen, Los Angeles, CA (US); Yu Jeong Kim, Los Angeles, CA (US)

(73) Assignee: University of Southern California, Los Angeles, CA (US)

(21) Appl. No.: 16/609,443

(22) PCT Filed: Jun. 22, 2018
(86) PCT No.: PCT/US18/39099

§ 371 Date: Oct. 29, 2019

Related U.S. Application Data
(60) Provisional application No. 62/523,401, filed on Jun. 22, 2017.

(51) Int. Cl. A61K 47/69; C12N 5/0783; C07K 16/28; C07K 16/32; A61K 35/17; A61K 9/00; A61P 35/00; A61K 31/337; C07K 14/705; C07K 14/725

(52) U.S. Cl. CPC A61K 47/6911; A61K 2039/545; A61K 47/6901; C07K 16/2803; C07K 16/32; A61K 35/17; A61K 9/0019; A61P 35/00; A61K 31/337; C07K 14/70521; C07K 14/70517; C07K 14/7051; C07K 14/70578; C12N 2510/00; C07K 2317/31; C07K 2319/03; C07K 2319/33; C07K 2317/24; C12N 5/0646

(57) ABSTRACT

Compositions are provided including NK cells that express chimeric antigen receptors (CARs) specific to CD19 and Her2 and a plurality of cell surface-bound multilamellar liposomal vesicles loaded with one or more anti-cancer therapeutics at an effective amount for inhibiting or killing tumor cells without causing toxicity to the NK cells. Methods of using these compositions to treat a subject with tumor are also provided, including administering an effective amount of the CAR-engineered NK cells, where an effective amount of anti-tumor therapeutics are delivered in particles (e.g., crosslinked multilamellar liposomal vesicles) that are bound to the surface of these CAR-engineered NK cells, without causing toxicity to the carrier NK cells.

### CROSS-REFERENCE TO RELATED APPLICATIONS

This application claims priority to and benefit of U.S. Provisional Application No. 62/523,401, filed on Jun. 22, 2017, the disclosure of which is hereby incorporated herein by reference in its entirety.

### STATEMENT REGARDING FEDERALLY SPONSORED RESEARCH OR DEVELOPMENT

This invention was made with government support under Grant No. AI068978 awarded by National Institutes of Health. The government has certain rights in the invention.

### FIELD OF THE INVENTION

Composition and methods for treating cancer are described herein.

### BACKGROUND OF THE INVENTION

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The therapeutic limitations of conventional chemotherapeutic drugs include chemo-resistance, tumor recurrence, and metastasis. Numerous nanoparticle-based active targeting approaches have emerged to enhance the intracellular concentration of drugs in tumor cells. However, efficient delivery of these systems to the tumor site while sparing healthy tissue remains elusive.

Recently, much attention has been given to human immune cell-directed nanoparticle drug delivery, as immune cells can traffic to the tumor and inflammatory sites. Natural killer cells are a subset of cytotoxic lymphocytes that play an important role in cancer immunosurveillance. Engineering of the human natural killer cell line, NK92, to express chimeric antigen receptors to redirect their antitumor specificity has shown significant promise.

Therefore it is an objective of the present invention to provide a composition and/or a delivery system that combines cell-based immunotherapy and chemotherapeutics for enhanced delivery, efficacy and specificity of anti-cancer therapies.

It is another objective of the present invention to provide a method of treating a subject with tumor by utilizing both immunotherapy and chemotherapeutics

### SUMMARY OF THE INVENTION

The following embodiments and aspects thereof are described and illustrated in conjunction with systems, compositions and methods which are meant to be exemplary and illustrative, not limiting in scope.

A chimeric antigen receptor (CAR)-engineered immune effector cell is provided to improve tumor-targeted delivery and efficacy, where the CAR contains an extracellular antigen specific domain and the cell surface is bound with a plurality of nano- or microparticles that contain an effective amount of active agents (e.g., chemotherapeutics) for efficacy against target cells without cytotoxicity to the carrier, CAR-engineered immune effector cell. Various embodiments provide that the immune effector cell is a natural killer (NK) cell. A CAR-engineered NK cells have polynucleotides encoding chimeric antigen receptors (CARs) or having expressed on the surface CARs. Generally, the bound particles are not endocytosed or internalized by the CAR-engineered NK cell, even though NK cells have phagocytotic capabilities. In some aspects, the CAR-engineered NK cells carry an effective amount of active agents (e.g., chemotherapeutics) to kill target cells without succumbing to chemotherapeutics-induced toxicity themselves. For example, an average effective amount of anti-tumor therapeutics that are delivered per CAR-engineered NK cell results in inhibition or killing of at least 10%, 20%, 30%, 40%, 50%, or 60% of targeted antigen-expressing tumor cells at a cell number ratio between 1:1 and 10:1 (e.g., 1:1, 2:1, 3:1, 4:1, 5:1, 6:1, 7:1, 8:1, 9:1 or 10:1) of engineered immune effector cell:antigen-expressing target tumor cell, but does not cause more than 1%, 3%, 5%, 7%, 10% or 15% cell death or cytotoxicity to the CAR-engineered NK cells. Other embodiments provide that the cell number ratio of engineered immune effector cell:antigen-expressing target tumor cell is greater than 10:1, e.g., 11:1, 12:1, 13:1, 14:1, 15:1, 20:1 25:1; which results in inhibition or killing of at least 10%, 20%, 30%, 40%, 50%, or 60% of targeted antigen-expressing tumor cells. Generally, in in vivo models, the dosage required of chemotherapeutic agents delivered via nanoparticles conjugated to the surface of NK cells to achieve inhibition of tumor growth and reduction of tumor size may be at least 8-fold, 9-fold, 10-fold, 11-fold, 12-fold, 13-fold, 14-fold, 15-fold, 20-fold, 30-fold, 40-fold, 50-fold, 100-fold or 200-fold less than the dosage required of free chemotherapeutic agents (without NK cells) for similar inhibition or reduction efficacy.

In various embodiments, the CAR-engineered NK cells having bound on the surface a plurality of active agent-loaded particles accumulate and have a higher concentration in the tumor environment following administration, and enhance antitumor efficacy, compared to that of CAR-engineered NK cells lacking surface-bound, active agent-loaded particles, whether these cells are administered alone or in a mixture with free, unbound active agent-loaded particles.

The conjugation of particles to the surface of CAR-engineered NK cells increases the release of type II interferon (IFN-γ) when the cells are cultured with target cells that have the cognate antigen (which the CAR recognizes), compared with the CAR-engineered NK cells that are cultured with target cells without the antigen. The released IFN-γ sensitizes tumor cells to NK cytotoxicity and initiates broad adaptive and innate immune responses.

In some embodiments, CAR-engineered NK92 cells are preferred to CAR-engineered T cells. NK92 cells proliferate in shorter time span than T cells, and are identical to parental cell line, thereby minimizing problems with donor variability. NK92 cells after irradiation are generally safe to use clinically, decreasing the risk of off-target effects compared to CAR-engineered T cells. Allogenic NK92 cell-based therapy including CAR engineering and particle conjugation, as disclosed herein, is generally less expensive than autologous CAR-engineering T cell-based similar therapy.

In one embodiment, the CAR binds to CD19. In another embodiment, the CAR binds Her2. In a further embodiment, the cell comprises bispecific CARs that bind CD19 and Her2. In some embodiments, the cells comprise the nucleic acids wherein the nucleic acids encode CARs that bind CD19 and Her2.

In some embodiments, the particles bound to the surface of CAR-engineered NK cells are nanoparticles, having an averaged diameter between 1 nm and 1,000 nm. In other embodiments, the particles bound to the surface of CAR-engineered NK cells are microparticles. Exemplary particles include liposomes or alternative liposomal formulations, such as cross-linked multilamellar liposomes (cMLV), and controlled release polymeric nanoparticles. In some aspects, cMLVs, as the active agent carrier, are bound to the surface of CAR-expressing NK cells, where interlipid bilayers are crosslinked in a liposome, resulting in a robust multilamellar structure. In other aspects, polymeric nanoparticles are the active agent carrier and bound to the surface of CAR-expressing NK cells. Depending on the solubility of the incorporated active agent, hydrophobic polymers or block copolymers may be selected, e.g., poly(lactic acid), poly(glycolic acid) or copolymer thereof, to form nanoparticles for controlled released of active agent therefrom. In one embodiment, cMLV are incubated with CAR-engineered NK cells at a number ratio greater than 500:1, e.g., about 1,000:1 or 2,000:1, to result in a conjugation ratio of about 100-150 cMLVs per CAR-engineered NK cell. In other embodiments depending on the size, chemical composition and linkage functional groups, the number of conjugated nanoparticles per CAR-engineered NK cell is between 400 and 350, between 350 and 300, between 300 and 250, between 250 and 200, between 200 and 150, or between 150 and 100. An exemplary conjugation chemistry is between maleimide group functionalized on the particles and free thiols on the immune effector cells. Optionally, a linker between the particles and the cell surface is present, e.g., via a polyethylene glycol.

Exemplary active agents include tumor therapeutics, such as paclitaxel and SN-38, pro-inflammatory cytokines, such as interleukin (IL)-15 and IL-21, check point inhibitors (e.g., PD-1 inhibitor including antibodies to PD-1), and immune-modulating agents. In one embodiment, the chemotherapeutic agent is paclitaxel. In another embodiment, two or more chemotherapeutic agents, such as paclitaxel and doxorubicin, are delivered in the same or individual nanoparticles that are bound to the surface of one CAR-expressing NK cell.

Also provided are pharmaceutical compositions comprising a NK cell containing nucleic acids encoding a chimeric antigen receptor (CAR), wherein the cell further contains on the surface bound crosslinked multilamellar liposomal vesicles (cMLVs) that encapsulate a chemotherapeutic agent; and a pharmaceutically acceptable carrier.

Further provided herein are methods for treating cancer in a subject in need thereof comprising administering to the subject an effective amount of the cells and/or pharmaceutically composition, as described herein.

A method of treating a subject with tumor(s) is also provided, wherein a composition including CAR-engineered NK cells (e.g., NK92 cells) with surface-bound nanoparticles is administered to the subject, the nanoparticles containing anti-cancer therapeutics, so as to enhance the delivery and efficacy of therapeutics and reduce off-target toxicity to normal tissue. Generally, the CAR is designed to bind an antigen of the cancer cells of the subject to which the composition is administered. For example, anti-CD19 CAR, anti-Her2 CAR, or both are expressed in the NK cells.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1A-FIG. 1D depict NK92 cell conjugation to maleimide-functionalized cMLVs. FIG. 1A shows a schematic of CAR.NK cells conjugated to PTX-loaded cMLVs. CARs are derived from the single chain variable fragment (scFv) of an antibody and the T cell receptor signaling complex. CARs can be transduced into NK92 cells and cMLVs can conjugate to the cell surface by interacting with free thiols. FIG. 1B shows cMLVs conjugated to the NK cell surface at various cMLV: cell ratios. cMLVs containing the fluorescent dye DiD were co-incubated with NK cells over a range of number ratios. The number of cMLVs on the surface of each cell was calculated by analyzing the DiD fluorescence. The ratio of 1000:1 provided the maximum amount of cMLVs per cell and was used in future experiments. FIG. 1C shows confocal microscopic images of CAR.NK cells conjugated to DiD-loaded cMLVs (cMLV(DiD)). CAR.NK cells were labeled with 1 μM CFSE and washed with PBS prior to conjugation to cMLV(DiD). Confocal microscopy was used to visualize the cMLVs on the CAR.NK cell surface. FIG. 1D shows an internalization assay of conjugated cMLVs. CAR.NK cells were conjugated with carboxyfluorescein-tagged maleimide-labeled cMLVs. The extracellular conjugation was quenched by trypan blue to differentiate surface-bound and internalized cMLVs 2 hours after conjugation. Attachment of cMLVs to CAR.NK-cells did not trigger the internalization of particles by the cells. Summarized statistics are displayed in the graphs (n=3, mean±SEM; NS, not significant; *p<0.05; **p<0.01; ***p<0.001).

FIG. 1E depicts CAR expression in transduced NK cells. Non-transduced NK cells were used as a negative control (gray shaded peaks). Anti-CD19 CARs were detected using flow cytometry after being labeled with biotinylated Protein L and streptavidin conjugated to FITC. Anti-Her2 CAR.NK cells were detected with flow cytometry after being labeled with rhHer2-Fc chimera and PE-labeled goat anti-human Fc.

FIG. 1F depicts confocal microscopy of CAR.NK cells conjugated to DiD-loaded cMLVs (cMLV(DiD))-3D and Z-stacked images. CAR.NK cells were labeled with 1 μM CFSE and washed with PBS prior to conjugation to cMLV(DiD).

FIG. 2A-FIG. 2B depict cytotoxicity of CAR.NK cells against CD19⁺ or Her2⁺ target cells. FIG. 2A shows cytotoxicity of anti-CD19 CAR.NK cells. Anti-CD19 CAR.NK cells were co-cultured with CD19⁻ SKOV3 cells or CD19⁺ SKOV.CD19 cells for 24 hours at 1:1, 5:1, or 10:1 effector-to-target ratios and cytotoxicity was measured. FIG. 2B shows cytotoxicity of anti-Her2 CAR.NK cells. Anti-Her2 CAR.NK cells were co-cultured with Her2⁻ MDA.MB.468 cells or Her2⁺ SKOV3 cells for 24 hours at 1:1, 5:1, or 10:1 effector-to-target ratios and cytotoxicity was measured. Summarized statistics are displayed in the graphs (n=3, mean±SEM; NS, not significant; *p<0.05; **p<0.01; ***p<0.001).

FIG. 2C depicts cytotoxicity comparison between irradiated (5 Gy) and nonirradiated CAR.NK cells. NK or CAR.NK cells were cocultured with SKOV.CD19 cells for 24 hours at 1:1, 5:1, or 10:1 effector-to-target ratios and cytotoxicity was measured.

FIG. 2D depicts cell viability assay with NK and SKOV3 cells exposed to PTX. Cells were incubated with various concentrations of cMLV(PTX). Cell viability percentage was determined by subtracting absorbance values obtained from media-only wells from the treated wells and then normalized by the control wells containing cells without drugs.

FIG. 2E depicts PTX release kinetics from free cMLVs and CAR.NK.cMLVs. To obtain the release kinetics of PTX from cMLVs before and after cell conjugation, cMLV(PTX) and CAR.NK.cMLV(PTX) were incubated in 10% FBS-containing media at 37° C. and were spun down and resuspended with fresh media daily. The PTX was quantified from the removed media by HPLC every day.

FIG. 3A-FIG. 3E depict CAR.NK cytokine release and migration when conjugated to cMLVs. FIG. 3A and FIG. 3B show IFNγ staining assays. Anti-CD19 (FIG. 3A) or anti-Her2 (FIG. 3B) CAR.NK cells were cocultured with various target cells with Brefeldin A protein transport inhibitor for 6 hours to detect IFNγ release. Unstimulated CAR.NK cells served as a negative control. CAR.NK cells were either unconjugated or conjugated with empty cMLVs (CAR.NK.cMLV(EMPTY)) or PTX-loaded cMLVs (CAR.NK.cMLV(PTX)). IFNγ was measured with intracellular staining. FIG. 3C and FIG. 3D show cytotoxicity assays. Anti-CD19 (FIG. 3C) or anti-Her2 (FIG. 3D) CAR.NK cells were cocultured with various target cells at a 1:1 ratio for 24 hours and cytotoxicity was measured. CAR.NK cells were either unconjugated or conjugated with empty cMLVs (CAR.NK.cMLV(EMPTY)) or PTX-loaded cMLVs (CAR.NK.cMLV(PTX)). FIG. 3E shows the migration assay. Unconjugated NK or NK conjugated to cMLV(EMPTY) were plated in the upper chambers of a Transwell plate. Negative controls had plain media in the lower wells, and CXCL9 was used as a chemoattractant in the lower wells of non-control groups. After 6 hours of incubation, media from the lower chambers was collected and NK cells were counted. Summarized statistics are displayed in the graphs (n=3, mean±SEM; NS, not significant; *p<0.05; **p<0.01; ***p<0.001).

FIG. 4A-FIG. 4F depict biodistribution of free cMLV(DiD) and conjugated CAR.NK.cMLV(DiD). Biodistribution data 24 hours (FIGS. 4A and 4B), 48 hours (FIGS. 4C and 4D), or 72 hours (FIGS. 4E and 4F) after intravenous injections. NOD/scid/IL2rγ−/− (NSG) mice bearing subcutaneous SKOV3.CD19 tumors were intravenously injected with 2×10⁷ CAR.NK cells conjugated with DiD-labeled cMLVs or an equivalent number of DiD-labeled cMLVs alone (n=3 per group per time point). After 24 hours, 48 hours, and 72 hours, indicated tissues were removed, weighed, and macerated with scissors. Specific DiD tissue fluorescence for each organ was quantified using the IVIS Spectrum imaging system and the mean percentage of injected dose per gram of tissue (% ID/g) was calculated as the final readout. Summarized statistics are displayed in the graphs (n=3, mean±SEM; NS, not significant; *p<0.05; **p<0.01; ***p<0.001).

FIG. 5 depicts antitumor efficacy of CAR.NK.cMLV(PTX) in solid tumor xenograft model. Tumor growth curve. SKOV.CD19 cells were injected subcutaneously into the right flank of NSG mice on Day 0. Mice were randomized into six groups (n=5 per group) and treated according to their group description four times total, 3-4 days apart via tail vein injection. Tumor size was measured with a fine caliper (n=5, mean±SEM; NS, not significant; *p<0.05; **p<0.01; ***p<0.001).

FIG. 6A-FIG. 6C depict ex vivo analysis of CAR.NK.cMLV(PTX) treatment. FIG. 6A shows the intratumoral PTX concentration. Thawed tumor samples were homogenized and PTX concentrations analyzed using HPLC (n=3, mean±SEM; NS, not significant; *p<0.05; **p<0.01; ***p<0.001). FIG. 6B shows TUNEL assay of fixed frozen tumor sections. Tumor sections were stained with a TUNEL kit according to the manufacturer's instructions and imaged with confocal microscopy. Representative images are shown herein. FIG. 6C shows the histology analysis for cardiac toxicity. Cardiac tissue was fixed and frozen, and sections were mounted on glass slides. The frozen sections were stained with hematoxylin and eosin. Histopathologic specimens were examined by light microscopy. Representative images are shown herein.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the term “about” refers to a measurable value such as an amount, a time duration, and the like, and encompasses variations of ±20%, ±10%, ±5%, ±1%, ±0.5% or ±0.1% from the specified value.

“Chimeric antigen receptor” or “CAR” or “CARs” as used herein refers to engineered receptors, which graft an antigen specificity onto cells (for example NK cells). CARs are also known as artificial T-cell receptors, chimeric T-cell receptors or chimeric immunoreceptors. In various embodiments, CARs are recombinant polypeptides comprising an antigen-specific domain (ASD), a hinge region (HR), a transmembrane domain (TMD), co-stimulatory domain (CSD) and an intracellular signaling domain (ISD).

“Effector function” refers to the specialized function of a differentiated cell. Effector function of a T-cell, for example, may be cytolytic/cytotoxicity activity or helper activity including the secretion of cytokines.

“Disease targeted by genetically modified cells” as used herein encompasses the targeting of any cell involved in any manner in any disease by the genetically modified cells of the invention, irrespective of whether the genetically modified cells target diseased cells or healthy cells to effectuate a therapeutically beneficial result. The genetically modified cells include but are not limited to genetically modified T-cells, NK cells, hematopoietic stem cells, pluripotent embryonic stem cells or embryonic stem cells. The genetically modified cells described herein express CARs that target specific antigens and in combination, function as chemotherapeutic drug delivery carriers. Examples of antigens which may be targeted include but are not limited to antigens expressed on B-cells; antigens expressed on carcinomas, sarcomas, lymphomas, leukemia, germ cell tumors, and blastomas; antigens expressed on various immune cells; and antigens expressed on cells associated with various hematologic diseases, autoimmune diseases, and/or inflammatory diseases. Other antigens that may be targeted will be apparent to those of skill in the art and may be targeted by the CARs of the invention in connection with alternate embodiments thereof.

“Autologous” cells as used herein refers to cells derived from the same individual as to whom the cells are later to be re-administered into.

“Genetically modified cells”, “redirected cells”, “genetically engineered cells”, “engineered cells” or “modified cells” as used herein refer to cells that express antigen-specific CARs and further have particles, preferably nanoparticles such as nano-sized liposomes (such as multilamellar liposomal vesicles) that carry a therapeutic agent such as a chemotherapeutic agent, where the particles are bound to the surface of the cells. In some embodiments, the genetically modified cells express CARs that target specific antigens and in combination, function as chemotherapeutic drug delivery carriers.

“Immune cell” as used herein refers to the cells of the mammalian immune system including but not limited to antigen presenting cells, B-cells, basophils, cytotoxic T-cells, dendritic cells, eosinophils, granulocytes, helper T-cells, leukocytes, lymphocytes, macrophages, mast cells, memory cells, monocytes, natural killer cells, neutrophils, phagocytes, plasma cells and T-cells.

The term “immune effector function” of the CAR-containing cell refers to any of the activities shown by the CAR-expressing cell upon stimulation by a stimulatory molecule. Examples of immune effector function, e.g., in a CAR-T cell, include cytolytic activity and helper activity, including the secretion of cytokines.

“Immune effector cell” as used herein includes the T cells and natural killer (NK) cells.

“Immune response” as used herein refers to immunities including but not limited to innate immunity, humoral immunity, cellular immunity, immunity, inflammatory response, acquired (adaptive) immunity, autoimmunity and/or overactive immunity.

As used herein, “CD4 lymphocytes” refer to lymphocytes that express CD4, i.e., lymphocytes that are CD4+. CD4 lymphocytes may be T cells that express CD4.

The terms “T-cell” and “T-lymphocyte” are interchangeable and used synonymously herein. Examples include but are not limited to naïve T cells, central memory T cells, effector memory T cells or combinations thereof.

As used herein, the term “antibody” refers to an intact immunoglobulin or to a monoclonal or polyclonal antigen-binding fragment with the Fc (crystallizable fragment) region or FcRn binding fragment of the Fc region, referred to herein as the “Fc fragment” or “Fc domain”. Antigen-binding fragments may be produced by recombinant DNA techniques or by enzymatic or chemical cleavage of intact antibodies. Antigen-binding fragments include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The Fc domain includes portions of two heavy chains contributing to two or three classes of the antibody. The Fc domain may be produced by recombinant DNA techniques or by enzymatic (e.g. papain cleavage) or via chemical cleavage of intact antibodies.

The term “antibody fragment,” as used herein, refer to a protein fragment that comprises only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)2 fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

“Single chain variable fragment”, “single-chain antibody variable fragments” or “scFv” antibodies as used herein refer to forms of antibodies comprising the variable regions of only the heavy (V_{H}) and light (V_{L}) chains, connected by a linker peptide. The scFvs are capable of being expressed as a single chain polypeptide. The scFvs retain the specificity of the intact antibody from which it is derived. The light and heavy chains may be in any order, for example, V_{H}-linker-V_{L} or V_{L}-linker-V_{H}, so long as the specificity of the scFv to the target antigen is retained.

“Complementarity determining region” (CDR) as used herein refers to the amino acid sequences within the variable regions of antibodies which regions confer specificity and binding affinity. In general, there are three CDRs in each of the light chain variable regions (LCDR1, LCDR2 and LCDR3) and three CDRs in each of the heavy chain variable regions (HCD1, HCDr2 and HCDR3). The boundaries of the CDRs may be determined using methods well known in the art including the “Kabat” numbering scheme and/or “Chothia” number scheme (Kabat et al. Sequences of Proteins of Immunological Interest, 5^{th} Ed. Public Health Services, National Institutes of Health, Bethesda, Md.; Al-Lazikani et al., (1997) JMB 273, 927-948).

As used herein, the term “specific binding” means the contact between an antibody and an antigen with a binding affinity of at least 10⁻⁶ M. In certain aspects, antibodies bind with affinities of at least about 10⁻⁷M, and preferably 10⁻⁸M, 10⁻⁹ M, 10¹⁰ M, 10⁻¹¹ M, or 10⁻¹² M.

“Therapeutic agents” as used herein refers to agents that are used to, for example, treat, inhibit, prevent, mitigate the effects of, reduce the severity of, reduce the likelihood of developing, slow the progression of and/or cure, a disease. Diseases targeted by the therapeutic agents include but are not limited to infectious diseases, cancers including but not limited to carcinomas, sarcomas, lymphomas, leukemia, germ cell tumors, and blastomas, antigens expressed on various immune cells, and antigens expressed on cells associated with various hematologic diseases, and/or inflammatory diseases.

“Cancer” and “cancerous” refer to or describe the physiological condition in mammals that is typically characterized by unregulated cell growth. The term “cancer” is meant to include all types of cancerous growths or oncogenic processes, metastatic tissues or malignantly transformed cells, tissues, or organs, irrespective of histopathologic type or stage of invasiveness. Examples of solid tumors include malignancies, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. In one embodiment, the cancer is a melanoma, e.g., an advanced stage melanoma. Metastatic lesions of the aforementioned cancers can also be treated or prevented using the methods and compositions of the invention. Examples of other cancers that can be treated include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. Treatment of metastatic cancers, e.g., metastatic cancers that express PD-L1 (Iwai et al. (2005) Int. Immunol. 17:133-144) can be effected using the antibody molecules described herein.

“Polynucleotide” as used herein includes but is not limited to DNA, RNA, cDNA (complementary DNA), mRNA (messenger RNA), rRNA (ribosomal RNA), shRNA (small hairpin RNA), snRNA (small nuclear RNA), snoRNA (short nucleolar RNA), miRNA (microRNA), genomic DNA, synthetic DNA, synthetic RNA, and/or tRNA.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both, cultured and engineered cells or tissues.

“Naked DNA” as used herein refers to DNA encoding a CAR cloned in a suitable expression vector in proper orientation for expression. Viral vectors which may be used include but are not limited SIN lentiviral vectors, retroviral vectors, foamy virus vectors, adeno-associated virus (AAV) vectors, hybrid vectors and/or plasmid transposons (for example sleeping beauty transposon system) or integrase based vector systems. Other vectors that may be used in connection with alternate embodiments of the invention will be apparent to those of skill in the art.

“Target cell” as used herein refers to cells which are involved in a disease and can be targeted by the genetically modified cells of the invention (including but not limited to genetically modified T-cells, NK cells, hematopoietic stem cells, pluripotent stem cells, and embryonic stem cells). Other target cells will be apparent to those of skill in the art and may be used in connection with alternate embodiments of the invention.

“Vector”, “cloning vector” and “expression vector” as used herein refer to the vehicle by which a polynucleotide sequence (e.g. a foreign gene) can be introduced into a host cell, so as to transform the host and promote expression (e.g. transcription and translation) of the introduced sequence. Vectors include plasmids, phages, viruses, etc.

As used herein, the term “administering,” refers to the placement an agent as disclosed herein into a subject by a method or route which results in at least partial localization of the agents at a desired site.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy. As non-limiting examples, “beneficial results” or “desired results” may be alleviation of one or more symptom(s), diminishment of extent of the deficit, stabilized (i.e., not worsening) state of cancer progression, delay or slowing of metastasis or invasiveness, and amelioration or palliation of symptoms associated with the cancer.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder, such as cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment). In some embodiments, treatment of cancer includes decreasing tumor volume, decreasing the number of cancer cells, inhibiting cancer metastases, increasing life expectancy, decreasing cancer cell proliferation, decreasing cancer cell survival, or amelioration of various physiological symptoms associated with the cancerous condition.

“Conditions” and “disease conditions,” as used herein may include, cancers, tumors or infectious diseases. In exemplary embodiments, the conditions include but are in no way limited to any form of malignant neoplastic cell proliferative disorders or diseases. In exemplary embodiments, conditions include any one or more of kidney cancer, melanoma, prostate cancer, breast cancer, glioblastoma, lung cancer, colon cancer, or bladder cancer.

The term “effective amount” or “therapeutically effective amount” as used herein refers to the amount of a pharmaceutical composition comprising one or more peptides as disclosed herein or a mutant, variant, analog or derivative thereof, to decrease at least one or more symptom of the disease or disorder, and relates to a sufficient amount of pharmacological composition to provide the desired effect. The phrase “therapeutically effective amount” as used herein means a sufficient amount of the composition to treat a disorder, at a reasonable benefit/risk ratio applicable to any medical treatment.

A therapeutically or prophylactically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject or the state of the subject prior to administering the oligopeptides described herein. Measured or measurable parameters include clinically detectable markers of disease, for example, elevated or depressed levels of a biological marker, as well as parameters related to a clinically accepted scale of symptoms or markers for diabetes. It will be understood, however, that the total daily usage of the compositions and formulations as disclosed herein will be decided by the attending physician within the scope of sound medical judgment. The exact amount required will vary depending on factors such as the type of disease being treated, gender, age, and weight of the subject.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment given for a disease”, which is often part of a standard set of treatments. When used by itself, first-line therapy is the one accepted as the best treatment. If it doesn't cure the disease or it causes severe side effects, other treatment may be added or used instead. It is also called induction therapy, primary therapy, and primary treatment. See National Cancer Institute website, last visited on Jun. 8, 2018. Typically, a patient is given a subsequent chemotherapy regimen because the patient did not show a positive clinical or sub-clinical response to the first line therapy or the first line therapy has stopped.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Particle” as used herein refers to particulate matters of various sizes and any shape. The appropriate particle size can vary based on the materials used to make the particle, the active agent or therapeutic agent carried therein, and the functional groups and chemistry involved for conjugation with an immune effector cell, as will be appreciated by a person of skill in the art in light of the teachings disclosed herein. For example, the particles can be nanoparticles having an averaged diameter between 1 nm and 1,000 nm, or microparticles having an averaged diameter greater than 1 μm but about at least an order of magnitude smaller than the immune effector cell to which the particles conjugated. For example, in some embodiments the particle has a diameter of from about 1 nm to about 1000 nm; or from about 25 nm to about 750 nm; or from about 50 nm to about 500 nm; or from about 100 nm to about 300 nm. In some embodiments, the average particle size can be about 1 nm, about 10 nm, about 50 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, or about 1000 nm, or about 2,000 nm, or about 5,000 nm, or about 6,000 nm, or about 10,000 nm. In some embodiments, the particle can be a nanoparticle or a microparticle, as these terms are defined herein. The particles can be all nanoparticles, all microparticles, or a combination of nanoparticles and microparticles. In some embodiments, the particles are liposomes. In other embodiments, the particles are polymeric particles formed from biocompatible and/or biodegradable polymers. In some embodiments, the particles contain a core. In some embodiments, the particles contain a coating.

“Biodegradable polymer” as used herein can contain a synthetic polymer, although natural polymers also can be used. The polymer can be, for example, poly(lactic-co-glycolic acid) (PLGA), polystyrene or combinations thereof. The polystyrene can, for example, be modified with carboxy groups. Other examples of biodegradable polymers include poly(hydroxy acid); poly(lactic acid); poly(glycolic acid); poly(lactic acid-co-glycolic acid); poly(lactide); poly(glycolide); poly(lactide-co-glycolide); polyanhydrides; polyorthoesters; polyamides; polycarbonates; polyalkylenes; polyethylene; polypropylene; polyalkylene glycols; poly(ethylene glycol); polyalkylene oxides; poly(ethylene oxides); polyalkylene terephthalates; poly(ethylene terephthalate); polyvinyl alcohols; polyvinyl ethers; polyvinyl esters; polyvinyl halides; poly(vinyl chloride); polyvinylpyrrolidone; polysiloxanes; poly(vinyl alcohols); poly(vinyl acetate); polyurethanes; co-polymers of polyurethanes; derivativized celluloses; alkyl cellulose; hydroxyalkyl celluloses; cellulose ethers; cellulose esters; nitro celluloses; methyl cellulose; ethyl cellulose; hydroxypropyl cellulose; hydroxy-propyl methyl cellulose; hydroxybutyl methyl cellulose; cellulose acetate; cellulose propionate; cellulose acetate butyrate; cellulose acetate phthalate; carboxylethyl cellulose; cellulose triacetate; cellulose sulfate sodium salt; polymers of acrylic acid; methacrylic acid; copolymers of methacrylic acid; derivatives of methacrylic acid; poly(methyl methacrylate); poly(ethyl methacrylate); poly(butylmethacrylate); poly(isobutyl methacrylate); poly(hexylmethacrylate); poly(isodecyl methacrylate); poly(lauryl methacrylate); poly(phenyl methacrylate); poly(methyl acrylate); poly(isopropyl acrylate); poly(isobutyl acrylate); poly(octadecyl acrylate); poly(butyric acid); poly(valeric acid); poly(lactide-co-caprolactone); copolymers of poly(lactide-co-caprolactone); blends of poly(lactide-co-caprolactone); poly-(isobutyl cyanoacrylate); poly(2-hydroxyethyl-L-glutamnine); and combinations, copolymers and/or mixtures of one or more of any of the foregoing. Furthermore, as a person of ordinary skill in the art would appreciate, some of the polymers listed above as “biocompatible” can also be considered biodegradable, whether or not they are included in the above listing of representative biodegradable polymers. As used herein, “derivatives” include polymers having substitutions, additions of chemical groups and other modifications routinely made by those skilled in the art.

“Cytotoxicity” refers to an agent being toxic to cells, which may be quantified as the extent of cell death (e.g., number of dead cells as a percentage of the original cell number before incubation with the agent) over a period of incubation time with the cells. For example, cytotoxicity is quantified as the number of dead cells as a percentage of the original cell number before incubation with the agent over 24 hours with the cells.

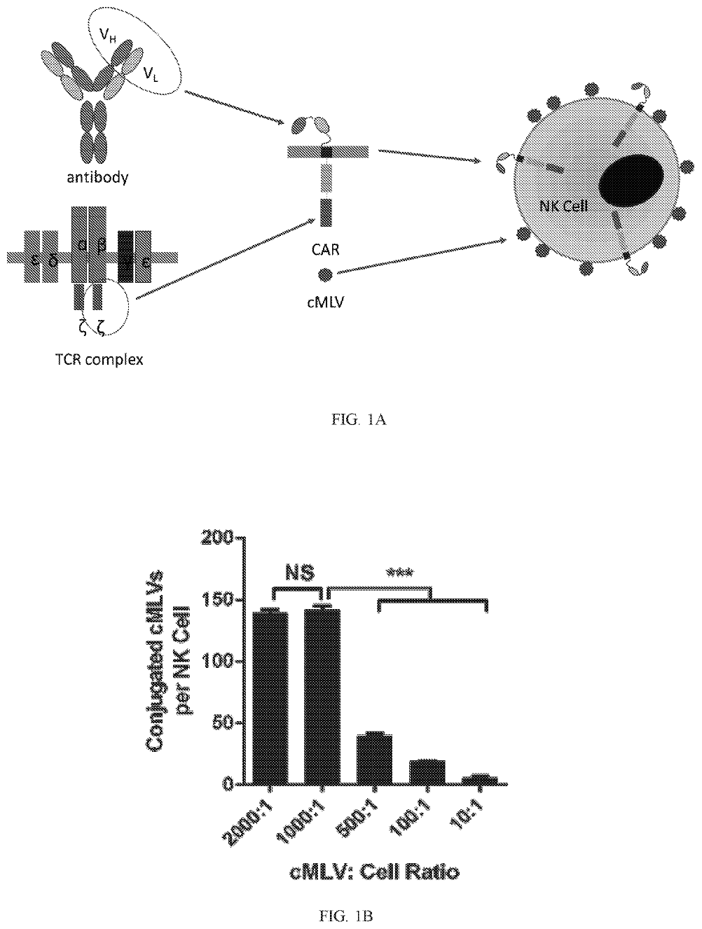

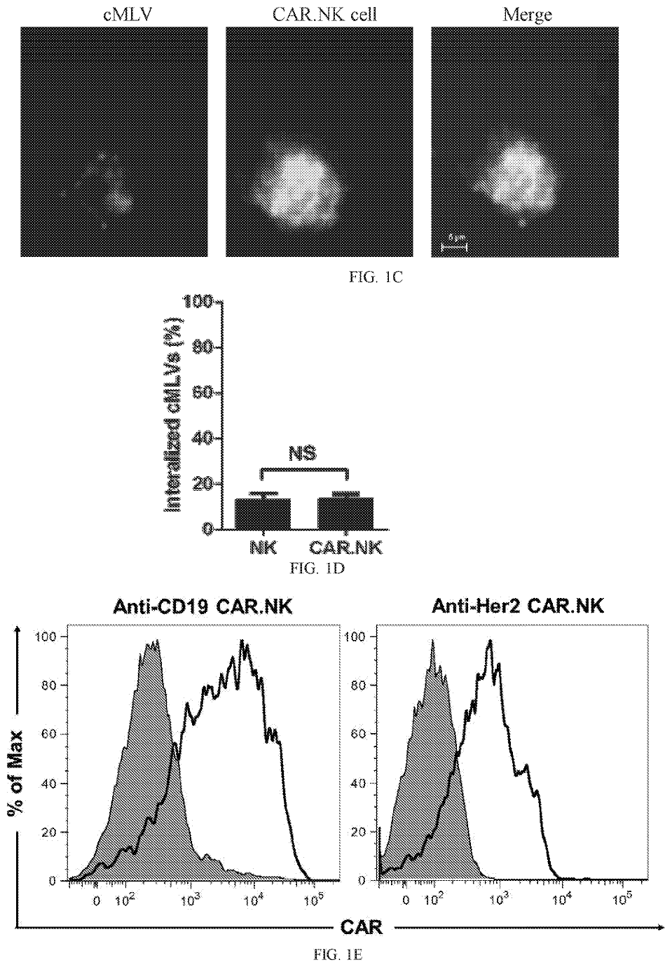

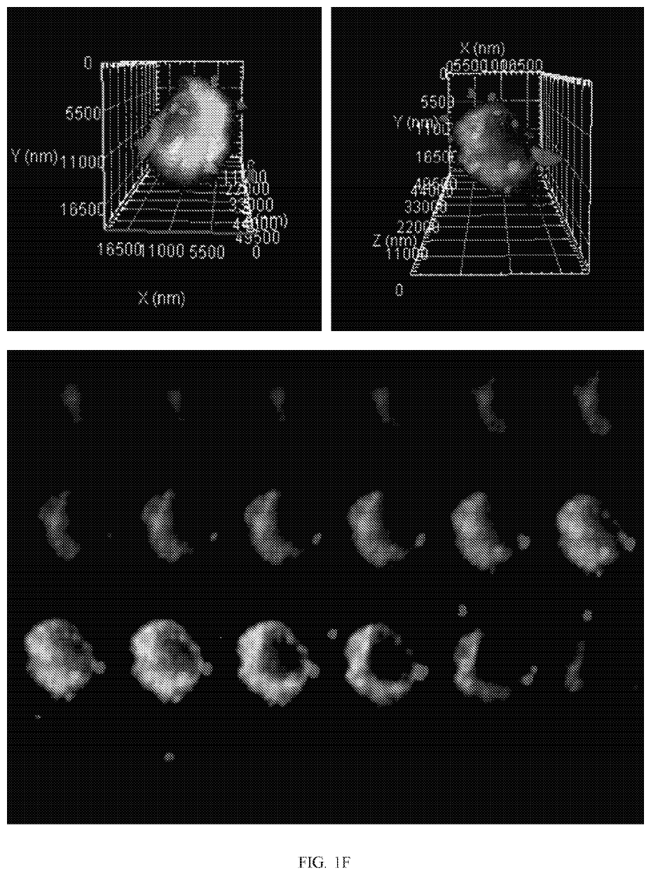

Without being bound to a particular theory, the efficacy of chemotherapy is enhanced as described in the present invention when tumor specific Natural Killer (NK) cells are used as carriers to deliver drug-loaded nanoparticles. In some embodiments, tumor-specific NK cells containing chimeric antigen receptors (CAR.NK) and crosslinked multilamellar liposomal vesicles (cMLVs) that encapsulate paclitaxel (PTX). In various aspects, these cMLVs are liposomes functionalized with thiol-reactive maleimide headgroups, which allow them to be stably conjugated to the thiol-rich NK cell surface. This composition and/or delivery system allows for combinatory drug delivery by co-localizing chemotherapeutics and immune effector cells to a single site (close proximity), inducing a synergistic anti-tumor effect in vitro and in vivo. Described herein is the combination of immunotherapy and chemotherapeutic drug delivery by utilizing CAR.NK cells as carriers for PTX-loaded crosslinked multilamellar liposomal vesicles (cMLV (PTX)) to enhance antitumor efficacy in Her2 and CD19 overexpressing cancer models (FIG. 1A).

### Composition

In some embodiments of the inventions, provided herein are genetically engineered cells which include vectors that express antigen-specific chimeric antigen receptors (CARs) and further include drug-loaded particles bound to the cell surface. In some embodiments, the particles are liposomes (e.g., crosslinked multilamellar vesicles) which are loaded with chemotherapeutic agents. In some embodiments, the CAR targets one antigen. In another embodiment, the CAR is a bispecific CAR and targets two different antigens. The bispecific CARs may target antigens on the same type of target cells or different cells.

In various embodiments, the genetically engineered cells expressing antigen-specific CARs and surface conjugated with therapeutics-loaded liposomes are T cells or Natural Killer (NK) cells. In one embodiment, the genetically engineered cells expressing CARs and surface conjugated with therapeutics-loaded liposomes are genetically engineered NK cells.

In various embodiments, the liposomes are multilamellar vesicle (with several lamellar phase lipid bilayers), small unilamellar liposome vesicle (with one lipid bilayer), large unilamellar vesicle or cochleate vesicle.

In some embodiments, the antigens which may be targeted by the CARs when expressed in cells (such as NK cells) as described herein include but are not limited to any one or more of CD19, CD22, CD23, MPL, CD123, CD32, CD138, CD200R, CD276, CD324, CD30, CD32, FcRH5, CD99, Tissue Factor, amyloid, Fc region of an immunoglobulin, CD171, CS-1, CLL-1 (CLECL1), CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, IL11Ra, Mesothelin, PSCA, VEGFR2, Lewis Y, CD24, PDGFR-beta, PRSS21, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLea, GM3, TGS5, BMWMAA, o-acetyl-GD2, Folate receptor beta, TEM1/CD248, TEM7R, CLDN6, TSHR, TCR-beta1 constant chain, TCR beta2 constant chain, TCR gamma-delta, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, legumain, HPV E6, E7, HTLV1-Tax, KSHV K8.1 protein, EBB gp350, HIV1-envelop glycoprotein gp120, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, DLL3, TROP2, PTK7, GCC, AFP, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, or combinations thereof.

In various embodiments, chemotherapeutic agents that may be encapsulated or otherwise delivered with the liposomes or polymeric particles include but are not limited to any one or more of Temozolomide, Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin, liposome-encapsulated Doxorubicin such as as Doxil (pegylated form), Myocet (nonpegylated form) and Caelyx, Epirubicin, Epothilone, Erlotinib, Etoposide, Fluorouracil, Folinic acid, Gefitinib, Gemcitabine, Hydroxyurea, Idarubicin, Imatinib, Ipilimumab, Irinotecan, Nanoliposomal Irinotecan (Nal-IRI), Mechlorethamine, Melphalan, Mercaptopurine, Methotrexate, Mitoxantrone, Ocrelizumab, Ofatumumab, Oxaliplatin, Paclitaxel, Taxol, Abraxane, Genexol, Protein-Bound Paclitaxel, Nab-Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, ixabepilone, Estramustine, prednisone, methylprednisolone, dexamethasone or a combination thereof.

In various embodiments, the chemotherapeutic agent is a platinum-based antineoplastic agent. Examples of the platinum-based antineoplastic agent include but are not limited to oxaliplatin, cisplatin, lipoplatin (a liposomal version of cisplatin), carboplatin, satraplatin, picoplatin, nedaplatin, and triplatin, and their functional equivalents, analogs, derivatives, variants or salts.

Generally, particles are conjugated to each cell at a ratio that does not negatively alter the function of the cell, yet high enough to deliver a high load of active agent per cell. For example, the number of conjugated nanoparticles (e.g., cMLVs) per cell is between 150 and 100, between 200 and 150, between 250 and 200, between 300 and 250, between 350 and 300, or between 400 and 350.

In some embodiments, the active agent, e.g., chemotherapeutics, in particles are delivered in an amount that does not cause cytotoxicity to the engineered NK cells following administration, yet high enough to inhibit or kill tumor cells in vitro and in vivo. In other embodiments, the active agent such as chemotherapeutics are carried in particles on CAR-expressing immune effector cells in an amount that causes cytotoxicity to less than 5%, 6%, 7%, 8%, 9%, 10%, 15%, or 20% of normal cells, yet high enough to inhibit or kill more than 10%, 15%, or 20% of tumor cells. In yet another embodiment, the active agent such as chemotherapeutics are carried in particles on CAR-expressing immune effector cells in an amount that causes cytotoxicity (e.g., death) to less than 5%, 6%, 7%, 8%, 9%, or 10% of a population of engineered NK cells, yet high enough to inhibit or kill more than 10%, 15%, or 20% of tumor cells.

In some embodiments, compositions are provided including genetically engineered NK cells, wherein the NK cells express one or more CARs and having bound on the surface crosslinked multilamellar liposomal vesicles (cMLVs) which encapsulate chemotherapeutic agents. In some embodiments, compositions are provided which includes genetically engineered NK cells, wherein the NK cells express CARs that target Her2 and are chemically bonded on the surface with a plurality of cMLVs that encapsulate chemotherapeutic agents. In some embodiments, provided herein are compositions including genetically engineered NK cells, wherein the NK cells express CARs that target CD19 and are chemically bonded on the surface with a plurality of cMLVs that encapsulate chemotherapeutic agents. In some embodiments, provided herein are compositions including genetically engineered NK cells, wherein the NK cells express CARs that target CD19 and Her2 and are chemically bonded on the surface with a plurality of cMLVs that encapsulate chemotherapeutic agents.

In some embodiments, provided herein are compositions including genetically engineered NK cells, wherein the NK cells express one or more CARs and are chemically bonded on the surface with a plurality of cMLVs that encapsulate paclitaxel. In some embodiments, provided herein are compositions including genetically engineered NK cells, wherein the NK cells express CARs that target Her2 and are chemically bonded on the surface with a plurality of cMLVs that encapsulate paclitaxel. In some embodiments, provided herein are compositions including genetically engineered NK cells, wherein the NK cells express CARs that target CD19 and are chemically bonded on the surface with a plurality of cMLVs that encapsulate paclitaxel. In some embodiments, provided herein are compositions including genetically engineered NK cells, wherein the NK cells express CARs that target CD19 and Her2 and are chemically bonded on the surface with a plurality of cMLVs that encapsulate paclitaxel.

Various embodiments provide crosslinked multilamellar liposomes as the active agent carrier, which are bound to the surface of an immune effector cell. A crosslinked multilamellar liposome has an exterior surface and an interior surface, the interior surface defining a central liposomal cavity. The multilamellar liposome includes at least a first lipid bilayer and a second lipid bilayer, the first lipid bilayer being covalently bonded to the second lipid bilayer. In one aspect, the lipid bilayers are covalently bonded by ether bonds and/or thioether bonds. Typically, multilamellar liposome includes at least one additional lipid bilayer such as third lipid bilayer which is covalently bonded to second lipid bilayer. In one embodiment, multilamellar liposome includes on average from 2 to 10 lipid bilayers. In another embodiment, multilamellar liposome includes on average from 3 to 9 lipid bilayers. In still another embodiment, multilamellar liposome includes on average from 3 to 6 lipid bilayers. In some variations, poly(alkylene glycol) groups (e.g., poly(ethylene glycol)) are covalently bonded to the exterior surface of the liposome in order to improve water solubility. For example, the poly(ethylene glycol) groups have a weight average molecular weight from about 400 to 2500 Daltons. In another refinement the poly(ethylene glycol) groups include from 9 to 45 repeat units of —OCH₂CH₂—.

Various chemical and/or physical interactions can be employed to bind a plurality of nanoparticles (e.g., cMLVs) to the surface of immune effector cells. In various embodiments, active agent-carrying nanoparticles are chemically bonded to the surface of the cell. In one embodiment, maleimide group is functionalized on the nanoparticles, which can chemically bond with the free thiols on the immune effector cells. Optionally, a linker between the nanoparticles and the cell surface is present, e.g., via a polyethylene glycol. Various embodiments provide that bound cMLVs on the surface of NK cells are not internalized or phagocytized by the NK cells.

Various embodiments provide at least one active agent, e.g., anticancer compound, is carried by a multilamellar liposome, through physical encapsulation, entrapment or chemical bonding. In one embodiment, an active agent can be disposed within the cavity of a crosslinked multilamellar liposome. In another embodiment, an active agent is disposed within the lipid bilayers and any additional lipid layers.

### Therapeutic Methods

Provided herein are methods for treating, inhibiting, preventing metastasis of and/or reducing severity of cancer in a subject in need thereof. The methods include administering to the subject an effective amount of a composition described herein.

In some embodiments, provided herein are methods for treating, inhibiting, preventing metastasis of and/or reducing severity of cancer in a subject in need thereof by administering to the subject an effective amount of a composition comprising genetically engineered NK cells which express CARs and are chemically bonded on the surface with a plurality of particles that encapsulate chemotherapeutic agents.

In some embodiments of the therapeutic methods described herein, the antigens which may be targeted by the CARs when expressed in cells (such as NK cells) as described herein include but are not limited to any one or more of CD19, CD22, CD23, MPL, CD123, CD32, CD138, CD200R, CD276, CD324, CD30, CD32, FcRH5, CD99, Tissue Factor, amyloid, Fc region of an immunoglobulin, CD171, CS-1, CLL-1 (CLECL1), CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, IL11Ra, Mesothelin, PSCA, VEGFR2, Lewis Y, CD24, PDGFR-beta, PRSS21, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLea, GM3, TGS5, HMWMAA, o-acetyl-GD2, Folate receptor beta, TEM1/CD248, TEM7R, CLDN6, TSHR, TCR-beta1 constant chain, TCR beta2 constant chain, TCR gamma-delta, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, legumain, HPV E6, E7, HTLV1-Tax, KSHV K8.1 protein, EBB gp350, HIV1-envelop glycoprotein gp120, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, DLL3, TROP2, PTK7, GCC, AFP, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, or combinations thereof.

In various embodiments of the therapeutic methods described herein, chemotherapeutic agents optionally in combination with other classes of compounds that may be encapsulated or otherwise delivered (e.g., including chemically bonded) with multilamellar liposomal vesicles include but are not limited to any one or more of Temozolomide, Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin, liposome-encapsulated Doxorubicin such as Doxil (pegylated form), Myocet (nonpegylated form) and Caelyx, Epirubicin, Epothilone, Erlotinib, Etoposide, Fluorouracil, Folinic acid, Gefitinib, Gemcitabine, Hydroxyurea, Idarubicin, Imatinib, Ipilimumab, Irinotecan, Nanoliposomal Irinotecan (Nal-IRI), Mechlorethamine, Melphalan, Mercaptopurine, Methotrexate, Mitoxantrone, Ocrelizumab, Ofatumumab, Oxaliplatin, Paclitaxel, Taxol, Abraxane, Genexol, Protein-Bound Paclitaxel, Nab-Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, ixabepilone, Estramustine, prednisone, methylprednisolone, dexamethasone or a combination thereof.

In one embodiment, the cancer specific antigen is expressed on both normal cells and cancers cells, but is expressed at lower levels on normal cells. In one embodiment, the method further comprises selecting a CAR that binds the cancer specific antigen of interest with an affinity that allows the antigen specific CAR to bind and kill the cancer cells. In some embodiments, the antigen specific CAR kills cancer cells but kills less than 30%, 25%, 20%, 15%, 10%, 5% or less of the normal cells expressing the cancer antigen. In exemplary embodiments, the percentage of cells killed by the antigen specific CARs may be determined using the cell death assays described herein.

In some embodiments, provided herein are methods for treating, inhibiting, preventing metastasis of and/or reducing severity of cancer in a subject in need thereof by administering to the subject an effective amount of a composition comprising NK cells that express CARs that target CD19 and the cells being chemically bonded on the surface with a plurality of cMLVs that encapsulate chemotherapeutic agents (e.g., paclitaxel).

In some embodiments, provided herein are methods for treating, inhibiting, preventing metastasis of and/or reducing severity of cancer in a subject in need thereof by administering to the subject an effective amount of a composition comprising NK cells that express CARs that target Her2 and the cells being chemically bonded on the surface with a plurality of cMLVs that encapsulate chemotherapeutic agents (e.g., paclitaxel).

In some embodiments, provided herein are methods for treating, inhibiting, preventing metastasis of and/or reducing severity of cancer in a subject in need thereof comprising administering to the subject an effective amount of a composition comprising NK cells that express CARs that target CD19 and Her2 and the cells being chemically bonded on the surface with a plurality of cMLVs that encapsulate chemotherapeutic agents (e.g., paclitaxel).

Exemplary cancers whose growth can be inhibited include cancers typically responsive to immunotherapy. Non-limiting examples of cancers for treatment include melanoma (e.g., metastatic malignant melanoma), renal cancer (e.g. clear cell carcinoma), prostate cancer (e.g. hormone refractory prostate adenocarcinoma), breast cancer, colon cancer and lung cancer (e.g. non-small cell lung cancer). Additionally, refractory or recurrent malignancies can be treated using the compositions described herein. In one embodiment, the engineered immune effector cell described herein is used for treatment of a subject with ovarian tumor. In another embodiment, the engineered immune effector cell described herein is used for treatment of a subject with melanoma. In yet another embodiment, the engineered immune effector cell described herein is used for treatment of a subject with renal cancer. Another embodiment provides the engineered immune effector cell described herein is used for treatment of a subject with prostate cancer. The engineered immune effector cell described herein can also be used for treatment of a subject with breast cancer, lung cancer, or both. Another embodiment provides the engineered immune effector cell described herein is used for treatment of a subject with leukemia.

In exemplary embodiments, cancers treated by the methods described herein include solid tumors such as sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. In one embodiment, the cancer is a melanoma, e.g., an advanced stage melanoma. Metastatic lesions of the aforementioned cancers can also be treated or prevented using the methods and compositions of the invention.

Examples of other cancers that can be treated include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. Treatment of metastatic cancers, e.g., metastatic cancers that express PD-L1 (Iwai et al. (2005) Int. Immunol. 17:133-144) can be effected using the antibody molecules described herein. Further a disease associated with a cancer associate antigen as described herein expression include, but not limited to, e.g., atypical and/or non-classical cancers, malignancies, precancerous conditions or proliferative diseases associated with expression of a cancer associate antigen as described herein. In some embodiments, a CAR-expressing T cell or NK cell as described herein reduces the quantity, number, amount or percentage of cells and/or cancer cells by at least 25%, at least 30%, at least 40%, at least 50%, at least 65%, at least 75%, at least 85%, at least 95%, or at least 99% in a subject with hematological cancer or another cancer associated with a cancer associated antigen as described herein, expressing cells relative to a negative control. In one embodiment, the subject is a human.

In some embodiments, the therapeutically effective amount of the genetically modified cells as described herein (for example, NK cells expressing CARs and further chemically bonded on the surface with a plurality of particles that encapsulate chemotherapeutic agents) is administered at a dosage of 10⁴ to 10⁹ cells/kg body weight, in some instances 10⁵ to 10⁶ cells/kg body weight, including all integer values within those ranges. In other instances, between about 0.1×10⁹ and 0.5×10⁹, between about 0.5×10⁹ and 1.0×10⁹, or between about 1.0×10⁹ and 5.0×10⁹ engineered immune effector cells are administered per injection to a human subject. Various embodiments provide that the immune effector cells are administered one or more times. Subsequent administrations typically occur at weekly, biweekly, triweekly, monthly, quarterly or yearly intervals, or at a combination of the frequencies mentioned above. T cell compositions may also be administered multiple times at these dosages. The cells can be administered by using infusion techniques that are commonly known in immunotherapy (see, e.g., Rosenberg et al., New Eng. J. of Med. 319:1676, 1988). The cells can be administered by injection into the site of the lesion (e.g., intra-tumoral injection).

In some embodiments, the therapeutic methods described herein further includes administering to the subject, sequentially or simultaneously, existing therapies. Examples of existing cancer treatment include, but are not limited to, active surveillance, observation, surgical intervention, chemotherapy, immunotherapy, radiation therapy (such as external beam radiation, stereotactic radiosurgery (gamma knife), and fractionated stereotactic radiotherapy (FSR)), focal therapy, systemic therapy, vaccine therapies, viral therapies, molecular targeted therapies, or combinations thereof.

Also provided herein are methods for preparing genetically engineered cells comprising transfecting the cells with vectors comprising nucleic acid encoding the CARs described herein. In some embodiments, the cells are immune effector cells, such as human T cells or human NK cells, or stem cells that give rise to immune effector cells. In some embodiments, the cells are autologous human T cells or autologous human NK cells or autologous human stem cells. In some embodiments, the cells are allogeneic human T cells or allogeneic human NK cells or allogeneic human stem cells.

In some embodiments, methods for preparing the genetically modified cells comprise obtaining a population of cells and selecting cells that express any one or more of CD3, CD28, CD4, CD8, CD45RA, and/or CD45RO. In certain embodiments, the population of immune effector cells provided are CD3+ and/or CD28+.

In one embodiment, the method for preparing the genetically modified cells comprise obtaining a population of cells and enriching for the CD25+T regulatory cells, for example by using antibodies specific to CD25. Methods for enriching CD25+T regulatory cells from the population of cells will be apparent to a person of skill in the art. In some embodiments, the Treg enriched cells comprise less than 30%, 20%, 10%, 5% or less non-Treg cells. In some embodiments, the vectors encoding the CARs described herein are transfected into Treg-enriched cells. Treg enriched cells expressing a CAR may be used to induced tolerance to antigen targeted by the CAR.

In some embodiments, the method further comprises expanding the population of cells after the vectors comprising nucleic acids encoding the CARs described herein have been transfected into the cells. In embodiments, the population of cells is expanded for a period of 8 days or less. In certain embodiments, the population of cells is expanded in culture for 5 days, and the resulting cells are more potent than the same cells expanded in culture for 9 days under the same culture conditions. In other embodiments, the population of cells is expanded in culture for 5 days show at least a one, two, three or four fold increase in cell doublings upon antigen stimulation as compared to the same cells expanded in culture for 9 days under the same culture conditions. In some embodiments, the population of cells is expanded in an appropriate media that includes one or more interleukins that result in at least a 200-fold, 250-fold, 300-fold, or 350-fold increase in cells over a 14 day expansion period, as measured by flow cytometry.

In various embodiments, the expanded cells comprise one or more CARs and further comprise liposomes (for example, multilamellar liposomal vesicles) conjugated to chemotherapeutic agents, as described herein. In some embodiments, the expanded cells comprise one CAR with one, two, three or more ASDs. In some embodiments, the expanded cells further comprise accessory modules and therapeutic controls as described herein.

### Combination Therapies

Therapeutic methods described herein include using compositions that have genetically modified cells which contain nucleic acids encoding CARs and are surface bonded with a plurality of chemotherapeutic agents-loaded particles. In various embodiments, the therapeutic methods described herein may be combined with existing therapies and agents. The therapeutic compositions described herein, e.g., genetically modified cells which contain nucleic acids encoding CARs and are surface bonded with a plurality of chemotherapeutic agents-loaded particles, are administered to the subject with at least one additional known therapy or therapeutic agent. In some embodiments, the compositions described herein and the additional therapy or therapeutic agents are administered sequentially. In some embodiments, the compositions described herein and the additional therapy or therapeutic agents are administered simultaneously. The optimum order of administering the compositions described herein and the existing therapies will be apparent to a person of skill in the art, such as a physician.

A genetically engineered CAR-expressing cell further including on the surface a plurality of chemotherapeutic agent-loaded nanoparticles, as described herein and the at least one additional therapeutic agent can be administered simultaneously, in the same or in separate compositions, or sequentially. For sequential administration, the cells described herein can be administered first, and the additional agent can be administered second, or the order of administration can be reversed.

Combinations therapies may be administered to the subject over the duration of the disease. Duration of the disease includes from diagnosis until conclusion of treatment, wherein the treatment results in reduction of symptoms and/or elimination of symptoms. In various embodiments, the effect of the two treatments can be partially additive, wholly additive, or greater than additive. The delivery can be such that an effect of the first treatment delivered is still detectable when the second is delivered.

Therapy using the cells described herein (for example, NK cells expressing CARs and further including on the surface a plurality of chemotherapeutic agent-loaded nanoparticles) and/or other therapeutic agents, procedures or modalities can be administered during periods of active disorder, or during a period of remission or less active disease. Therapy using the cells described herein (for example, NK cells expressing CARs and further including on the surface a plurality of chemotherapeutic agent-loaded cMLVs) can be administered before the other treatment, concurrently with the treatment, post-treatment, or during remission of the disorder.

When administered in combination, the therapy using the cells described herein (for example, NK cells expressing CARs and further including on the surface a plurality of chemotherapeutic agent-loaded cMLVs) and the additional agent (e.g., second or third agent), or all, can be administered in an amount or dose that is higher, lower or the same than the amount or dosage of each agent used individually, e.g., as a monotherapy. In certain embodiments, the administered amount or dosage of the therapy using the cells described herein (for example, NK cells expressing CARs and further including on the surface a plurality of chemotherapeutic agent-loaded cMLVs), the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the therapy using the cells described herein (for example, NK cells expressing CARs and further including on the surface a plurality of chemotherapeutic agent-loaded cMLVs), the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

Further method aspects relate administering to the subject an effective amount of the cell described herein (for example, NK cells expressing CARs and further including on the surface a plurality of chemotherapeutic agent-loaded cMLVs), optionally in combination with an agent that increases the efficacy and/or safety of the immune cell. In further aspects, the agent that increases the efficacy and/or safety of the immune cell is one or more of: (i) a protein phosphatase inhibitor; (ii) a kinase inhibitor; (iii) a cytokine; (iv) an inhibitor of an immune inhibitory molecule; or (v) an agent that decreases the level or activity of a TREG cell; vi) an agent that increase the proliferation and/or persistence of CAR-modified cells vii) a chemokine viii) an agent that increases the expression of CAR ix) an agent that allows regulation of the expression or activity of CAR x) an agent that allows control over the survival and/or persistence of CAR-modified cells xi) an agent that controls the side effects of CAR-modified cells xii) a Brd4 inhibitor xiii) an agent that delivers a therapeutic (e.g. sHVEM) or prophylactic agent to the site of the disease xiv) an agent that increases the expression of the target antigen against which CAR is directed; xv) an adenosine A2a receptor antagonist

In some embodiments, the genetically modified cells described herein (for example, NK cells expressing CARs and further including on the surface a plurality of chemotherapeutic agent-loaded cMLVs) may be used in a treatment regimen in combination with surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation, peptide vaccine, such as that described in Izumoto et al. 2008 J Neurosurg 108:963-971. In one embodiment, a CAR-expressing cell described herein can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

In embodiments, the cell described herein (for example, NK cells expressing CARs and including on the surface a plurality of chemotherapeutic agent-loaded cMLVs) is administered to a subject in combination with cyclophosphamide and fludarabine.

In embodiments, the cell described herein (for example, NK cells expressing CARs and further including on the surface a plurality of chemotherapeutic agent-loaded cMLVs) is administered to a subject in combination with bendamustine and rituximab. In embodiments, the subject has CLL.

In embodiments, the cell described herein (for example, NK cells expressing CARs and further including on the surface a plurality of chemotherapeutic agent-loaded cMLVs) is administered to a subject in combination with rituximab, cyclophosphamide, doxorubicin, vincristine, and/or a corticosteroid (e.g., prednisone). In embodiments, a CAR-expressing cell described herein is administered to a subject in combination with rituximab, cyclophosphamide, doxorubicin, vincristine, and prednisone (R-CHOP). In embodiments, the subject has diffuse large B-cell lymphoma (DLBCL).

In embodiments, the cell described herein (for example, NK cells expressing CARs and further including on the surface a plurality of chemotherapeutic agent-loaded cMLVs) is administered to a subject in combination with etoposide, prednisone, vincristine, cyclophosphamide, doxorubicin, and/or rituximab. In embodiments, a CAR-expressing cell described herein is administered to a subject in combination with etoposide, prednisone, vincristine, cyclophosphamide, doxorubicin, and rituximab (EPOCH-R). In embodiments, a CAR-expressing cell described herein is administered to a subject in combination with dose adjusted EPOCH-R (DA-EPOCH-R). In embodiments, the subject has a B cell lymphoma, e.g., a Myc-rearranged aggressive B cell lymphoma.

In embodiments, the described herein (for example, NK cells expressing CARs and further including on the surface a plurality of chemotherapeutic agent-loaded cMLVs) is administered to a subject in combination with brentuximab. Brentuximab is an antibody-drug conjugate of anti-CD30 antibody and monomethyl auristatin E. In embodiments, the subject has Hodgkin's lymphoma (HL), e.g., relapsed or refractory HL. In embodiments, the subject comprises CD30+HL. In embodiments, the subject has undergone an autologous stem cell transplant (ASCT).

In some embodiments, the cell described herein (for example, NK cells expressing CARs and further including on the surface a plurality of chemotherapeutic agent-loaded cMLVs) is administered to a subject in combination with a CD20 inhibitor, e.g., an anti-CD20 antibody (e.g., an anti-CD20 mono- or bispecific antibody) or a fragment thereof.

In one embodiment, the described herein (for example, NK cells expressing CARs and further including on the surface a plurality of chemotherapeutic agent-loaded cMLVs) is administered to a subject in combination with an mTOR inhibitor, e.g., an mTOR inhibitor described herein, e.g., a rapalog such as everolimus. In one embodiment, the mTOR inhibitor is administered prior to the CAR-expressing cell. For example, in one embodiment, the mTOR inhibitor can be administered prior to apheresis of the cells. In one embodiment, the subject has CLL.

In one embodiment, the cell described herein (for example, NK cells expressing CARs and further including on the surface a plurality of chemotherapeutic agent-loaded cMLVs) can be used in combination with a kinase inhibitor. In one embodiment, the kinase inhibitor is a CDK4 inhibitor, e.g., a CDK4 inhibitor described herein, e.g., a CD4/6 inhibitor, such as, e.g., 6-Acetyl-8-cyclopentyl-5-methyl-2-(5-piperazin-1-yl-pyridin-2-ylamino)-8H-pyrido[2,3-d]pyrimidin-7-one, hydrochloride (also referred to as palbociclib or PD0332991). In one embodiment, the kinase inhibitor is a BTK inhibitor, e.g., a BTK inhibitor described herein, such as, e.g., ibrutinib. In some embodiments, ibrutinib is administered at a dosage of about 300-600 mg/day (e.g., about 300-350, 350-400, 400-450, 450-500, 500-550, or 550-600 mg/day, e.g., about 420 mg/day or about 560 mg/day), e.g., orally. In embodiments, the ibrutinib is administered at a dose of about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) daily for a period of time, e.g., daily for 21 day cycle, or daily for 28 day cycle. In one embodiment, 1, 2, 3, 4, 5, 6, 7, 8, 9, 10, 11, 12 or more cycles of ibrutinib are administered. Without being bound by theory, it is thought that the addition of ibrutinib enhances the T cell proliferative response and may shift T cells from a T-helper-2 (Th2) to T-helper-1 (Th1) phenotype. Th1 and Th2 are phenotypes of helper T cells, with Th1 versus Th2 directing different immune response pathways. A Th1 phenotype is associated with proinflammatory responses, e.g., for killing cells, such as intracellular pathogens/viruses or cancerous cells, or perpetuating autoimmune responses. A Th2 phenotype is associated with eosinophil accumulation and anti-inflammatory responses. In one embodiment, the kinase inhibitor is an mTOR inhibitor, e.g., an mTOR inhibitor described herein, such as, e.g., rapamycin, a rapamycin analog, OSI-027. The mTOR inhibitor can be, e.g., an mTORC1 inhibitor and/or an mTORC2 inhibitor, e.g., an mTORC1 inhibitor and/or mTORC2 inhibitor described herein. In one embodiment, the kinase inhibitor is a MNK inhibitor, e.g., a MNK inhibitor described herein, such as, e.g., 4-amino-5-(4-fluoroanilino)-pyrazolo[3,4-d] pyrimidine. The MNK inhibitor can be, e.g., a MNK1a, MNK1b, MNK2a and/or MNK2b inhibitor. In one embodiment, the kinase inhibitor is a dual PI3K/mTOR inhibitor described herein, such as, e.g., PF-04695102. In one embodiment, the kinase inhibitor is a Src kinase inhibitor. In one embodiment, the kinase inhibitor is Dasatinib. In one embodiment, the Src kinase inhibitor is administered to the patient after the administration of CAR expressing cells to control or terminate the activity of CAR-expressing cells. In one embodiment, Dasatinib is administered to the patient after the administration of CAR expressing cells to control or terminate the activity of CAR-expressing cells. In one embodiment, dasatinib is administered orally at a dose of at least 10 mg/day, 20 mg/day, 40 mg/day, 60 mg/day, 70 mg/day, 90 mg/day, 100 mg/day, 140 mg/day, 180 mg/day or 210 mg/day.

In embodiments, the cell described herein (for example, NK cells expressing CARs and further including on the surface a plurality of chemotherapeutic agent-loaded cMLVs) is administered to a subject in combination with an anaplastic lymphoma kinase (ALK) inhibitor. Exemplary ALK kinases include but are not limited to crizotinib (Pfizer), ceritinib (Novartis), alectinib (Chugai), brigatinib (also called AP26113; Ariad), entrectinib (Ignyta), PF-06463922 (Pfizer), TSR-011 (Tesaro) (see, e.g., Clinical Trial Identifier No. NCT02048488), CEP-37440 (Teva), and X-396 (Xcovery). In some embodiments, the subject has a solid cancer, e.g., a solid cancer described herein, e.g., lung cancer.

Drugs that inhibit either the calcium dependent phosphatase calcineurin (cyclosporine and FK506) or inhibit the p70S6 kinase that is important for growth factor induced signaling (rapamycin) can also be used. In a further aspect, the cell compositions of the present invention may be administered to a patient in conjunction with (e.g., before, simultaneously or following) bone marrow transplantation, T cell ablative therapy using chemotherapy agents such as, fludarabine, external-beam radiation therapy (XRT), cyclophosphamide, and/or antibodies such as OKT3 or CAMP ATH. In one aspect, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

In embodiments, the cell described herein (for example, NK cells expressing CARs and further including on the surface a plurality of chemotherapeutic agent-loaded cMLVs) is administered to a subject in combination with an autologous stem cell transplant, an allogeneic stem cell transplant, an autologous bone marrow transplant or an allogeneic bone marrow transplant.

In embodiments, the cell described herein (for example, NK cells expressing CARs and including on the surface a plurality of chemotherapeutic agent-loaded cMLVs) is administered to a subject in combination with microtransplant or HLA mismatched allogeneic cellular therapy.

In embodiments, the cell described herein (for example, NK cells expressing CARs and further including on the surface a plurality of chemotherapeutic agent-loaded cMLVs) is administered to a subject in combination with an indoleamine 2,3-dioxygenase (IDO) inhibitor.

In embodiments, the cell described herein (for example, NK cells expressing CARs and further including on the surface a plurality of chemotherapeutic agent-loaded cMLVs) is administered to a subject in combination with a modulator of myeloid-derived suppressor cells (MDSCs). MDSCs accumulate in the periphery and at the tumor site of many solid tumors. These cells suppress T cell responses, thereby hindering the efficacy of CAR-expressing cell therapy. Without being bound by theory, it is thought that administration of a MDSC modulator enhances the efficacy of a CAR-expressing cell described herein. In an embodiment, the subject has a solid tumor, e.g., a solid tumor described herein, e.g., glioblastoma. Exemplary modulators of MDSCs include but are not limited to MCS11O and BLZ945. MCS11O is a monoclonal antibody (mAb) against macrophage colony-stimulating factor (M-CSF). BLZ945 is a small molecule inhibitor of colony stimulating factor 1 receptor (CSF1R). In embodiments, the cell described herein (for example, NK cells expressing CARs and further including on the surface a plurality of chemotherapeutic agent-loaded cMLVs) is administered to a subject in combination with a Brd4 or BET (bromodomain and extra-terminal motif) inhibitor. BET protein BRD4 directly regulated expression of the transcription factor BATF in CD8+ T cells, which was associated with differentiation of T cells into an effector memory phenotype. JQ1, an inhibitor of bromodomain and extra-terminal motif (BET) proteins, maintained CD8+ T cells with functional properties of stem cell-like and central memory T cells. Exemplary Brd4 inhibitors that can be administered in combination with CAR-expressing cells include but are not limited to JQ1, MS417, OTXO15, LY 303511 and Brd4 inhibitor as described in US 20140256706 A1 and any analogs thereof.

In some embodiments, the cell described herein (for example, NK cells expressing CARs and further including on the surface a plurality of chemotherapeutic agent-loaded cMLVs) is administered to a subject in combination with a interleukin-15 (IL-15) polypeptide, a interleukin-15 receptor alpha (IL-15Ra) polypeptide, or a combination of both a IL-15 polypeptide and a IL-15Ra polypeptide e.g., hetiL-15 (Admune Therapeutics, LLC). hetiL-15 is a heterodimeric non-covalent complex of IL-15 and IL-15Ra. hetiL-15 is described in, e.g., U.S. Pat. No. 8,124,084, U.S. 2012/0177598, U.S. 2009/0082299, U.S. 2012/0141413, and U.S. 201110081311. In embodiments, het-IL-15 is administered subcutaneously. In embodiments, the subject has a cancer, e.g., solid cancer, e.g., melanoma or colon cancer. In embodiments, the subject has a metastatic cancer.

In one embodiment, the subject can be administered an agent which reduces or ameliorates a side effect associated with the administration of a CAR-expressing cell. Side effects associated with the administration of a CAR-expressing cell include, but are not limited to CRS, and hemophagocytic lymphohistiocytosis (HLH), also termed Macrophage Activation Syndrome (MAS). Symptoms of CRS include high fevers, nausea, transient hypotension, hypoxia, and the like. CRS may include clinical constitutional signs and symptoms such as fever, fatigue, anorexia, myalgias, arthalgias, nausea, vomiting, and headache. CRS may include clinical skin signs and symptoms such as rash. CRS may include clinical gastrointestinal signs and symptoms such as nausea, vomiting and diarrhea. CRS may include clinical respiratory signs and symptoms such as tachypnea and hypoxemia. CRS may include clinical cardiovascular signs and symptoms such as tachycardia, widened pulse pressure, hypotension, increased cardiac output (early) and potentially diminished cardiac output (late). CRS may include clinical coagulation signs and symptoms such as elevated d-dimer, hypofibrinogenemia with or without bleeding. CRS may include clinical renal signs and symptoms such as azotemia. CRS may include clinical hepatic signs and symptoms such as transaminitis and hyperbilirubinemia. CRS may include clinical neurologic signs and symptoms such as headache, mental status changes, confusion, delirium, word finding difficulty or frank aphasia, hallucinations, tremor, dymetria, altered gait, and seizures.

Accordingly, the methods described herein can include administering the cell described herein (for example, NK cells expressing CARs and including on the surface a plurality of chemotherapeutic agent-loaded nanoparticles such as cMLVs) to a subject and further administering one or more agents to manage elevated levels of a soluble factor resulting from treatment with a CAR-expressing cell. In one embodiment, the soluble factor elevated in the subject is one or more of IFN-γ, TNFa, IL-2 and IL-6. In an embodiment, the factor elevated in the subject is one or more of IL-1, GM-CSF, IL-10, IL-8, IL-5 and fraktalkine. Therefore, an agent administered to treat this side effect can be an agent that neutralizes one or more of these soluble factors. In one embodiment, the agent that neutralizes one or more of these soluble forms is an antibody or antigen binding fragment thereof. Examples of such agents include, but are not limited to a steroid (e.g., corticosteroid), Src inhibitors (e.g., Dasatinib) an inhibitor of TNFa, and an inhibitor of IL-6. An example of a TNFa inhibitor is an anti-TNFa antibody molecule such as, infliximab, adalimumab, certolizumab pegol, and golimumab. Another example of a TNFa inhibitor is a fusion protein such as entanercept. An example of an IL-6 inhibitor is an anti-IL-6 antibody molecule or an anti-IL-6 receptor antibody molecule such as tocilizumab (toe), sarilumab, elsilimomab, CNTO 328, ALD518/BMS-945429, CNTO 136, CPSI-2364, CDP6038, VX30, ARGX-109, FE301, and FM101. In one embodiment, the anti-IL-6 receptor antibody molecule is tocilizumab. In one embodiment, the IL-6 inhibitor is a camelid bispecific antibody that binds to IL6R and human serum albumin (e.g., IL6R-304-Alb8) (SEQ ID NO: 2649). An example of an IL-1R based inhibitor is anakinra. In one embodiment, an agent administered to treat the side effects of CAR-expressing cells is a Src inhibitor (e.g., Dasatinib). In one embodiment, an agent administered to treat the side effects of CAR-expressing cells is the Src inhibitor Dasatinib. In embodiments, Dasatinib is administered at a dose of about 10 mg/day to 240 mg/day (e.g., 10 mg/day, 20 mg/day, 40 mg/day, 50 mg/day, 70 mg/day, 80 mg/day, 100 mg/day, 110 mg/day, 120 mg/day, 140 mg/day, 180 mg/day, 210 mg/day, 240 mg/day or 300 mg/day).

In one embodiment, the subject can be administered an agent which enhances the activity of the cell described herein (for example, NK cells expressing CARs and further including on the surface a plurality of chemotherapeutic agent-loaded nanoparticles such as cMLVs). For example, in one embodiment, the agent can be an agent which inhibits an inhibitory molecule. Inhibitory molecules, e.g., Programmed Death 1 (PD-1), can, in some embodiments, decrease the ability of a CAR-expressing cell to mount an immune effector response. Examples of inhibitory molecules include PD-1, PDL1, CTLA-4, TIM-3, CEACAM (e.g., CEACAM-1, CEACAM-3 and/or CEACAM-5), LAG-3, VISTA, BTLA, TIGIT, LAIR1, CD160, 2B4 and TGFR beta. Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used to inhibit expression of an inhibitory molecule in the CAR-expressing cell. In an embodiment the inhibitor is an shRNA. In an embodiment, the inhibitory molecule is inhibited within a CAR-expressing cell. In these embodiments, a dsRNA molecule that inhibits expression of the inhibitory molecule is linked to the nucleic acid that encodes a component, e.g., all of the components, of the CAR. In one embodiment, the inhibitor of an inhibitory signal can be, e.g., an antibody or antibody fragment that binds to an inhibitory molecule. For example, the agent can be an antibody or antibody fragment that binds to PD-1, PD-L1, PD-L2 or CTLA4 (e.g., ipilimumab (also referred to as MDX-010 and MDX-101, and marketed as Yervoy®; Bristol-Myers Squibb; Tremelimumab (IgG2 monoclonal antibody available from Pfizer, formerly known as ticilimumab, CP-675,206).). In an embodiment, the agent is an antibody or antibody fragment that binds to TIM3. In an embodiment, the agent is an antibody or antibody fragment that binds to CEACAM (CEACAM-1, CEACAM-3, and/or CEACAM-5). In an embodiment, the agent is an antibody or antibody fragment that binds to LAG3.

PD-1 is an inhibitory member of the CD28 family of receptors that also includes CD28, CTLA-4, ICOS, and BTLA. PD-1 is expressed on activated B cells, T cells and myeloid cells. Two ligands for PD-1, PD-L1 and PD-L2 have been shown to down regulate T cell activation upon binding to PD-1. PD-L1 is abundant in human cancers. Immune suppression can be reversed by inhibiting the local interaction of PD-1 with PD-L1. Antibodies, antibody fragments, and other inhibitors of PD-1, PD-L1 and PD-L2 are available in the art and may be used combination with a CAR of the present invention described herein. For example, nivolumab (also referred to as BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449 and WO2006/121168. Pidilizumab (CT-011; Cure Tech) is a humanized IgG1k monoclonal antibody that binds to PD-1. Pidilizumab and other humanized anti-PD-1 monoclonal antibodies are disclosed in WO2009/101611. Pembrolizumab (formerly known as lambrolizumab, and also referred to as MK03475; Merck) is a humanized IgG4 monoclonal antibody that binds to PD-1. Pembrolizumab and other humanized anti-PD-1 antibodies are disclosed in U.S. Pat. No. 8,354,509 and WO2009/114335. MEDI4736 (Medimmune) is a human monoclonal antibody that binds to PDL1, and inhibits interaction of the ligand with PD1. MDPL3280A (Genentech I Roche) is a human Fe optimized IgG1 monoclonal antibody that binds to PD-L1. MDPL3280A and other human monoclonal antibodies to PD-L1 are disclosed in U.S. Pat. No. 7,943,743 and U.S Publication No.: 20120039906. In other embodiments, the agent that enhances the activity of a CAR-expressing cell is a CEACAM inhibitor (e.g., CEACAM-1, CEACAM-3, and/or CEACAM-5 inhibitor).

In one embodiment, the agent that enhances activity of the cell described herein (for example, NK cells expressing CARs and further including on the surface a plurality of chemotherapeutic agent-loaded nanoparticles such as cMLVs) is another agent that increases the expression of the target antigen against which the CAR is directed. The agents that can be administered to the subject receiving a CAR-expressing cell described herein include: Arsenic trioxide, ATRA (all-trans-retinoic acid), compounds 27, 40, 49 of, IDH2 inhibitors (e.g., AG-221) or a combination thereof. In an embodiment, the agents are administered prior to, concurrently or after administration of CAR-expressing cells. In preferred embodiments these agents are administered prior to administration of CAR-expressing cells. In preferred embodiment, the CAR expressing cells that are administered with the above agents target a B cell antigen (e.g., CD19, CD20, or CD22 etc.).

Cytokines that can be administered to the subject receiving the cell described herein (for example, NK cells expressing CARs and further including on the surface a plurality of chemotherapeutic agent-loaded nanoparticles such as cMLVs) include: IL-2, IL-4, IL-7, IL-9, IL-15, IL-18, LIGHT, and IL-21, or a combination thereof. In preferred embodiments, the cytokine administered is IL-7, IL-15, or IL-21, IL12F, or a combination thereof. The cytokine can be administered once a day or more than once a day, e.g., twice a day, three times a day, or four times a day. The cytokine can be administered for more than one day, e.g. the cytokine is administered for 2 days, 3 days, 4 days, 5 days, 6 days, 1 week, 2 weeks, 3 weeks, or 4 weeks. For example, the cytokine is administered once a day for 7 days. Administration of the cytokine to the subject that has sub-optimal response to the CAR-expressing cell therapy improves CAR-expressing cell efficacy or anti-cancer activity. In a preferred embodiment, the cytokine administered after administration of CAR-expressing cells is IL-7.

In one embodiment, the agent which enhances activity of the cell described herein (for example, NK cells expressing CARs and further including on the surface a plurality of chemotherapeutic agent-loaded nanoparticles such as cMLVs) is a Brd4 inhibitor or an siRNA or an shRNA targeting BRD4.

### Pharmaceutical Composition

In various embodiments, the present invention provides a pharmaceutical composition. The pharmaceutical composition includes genetically modified cells expressing antigen-specific CARs and having bound on the cell surface a plurality of liposomes (for example, multilamellar liposomal vesicles) or other naonparticles which encapsulate or otherwise carry one or more chemotherapeutic agents; and any pharmaceutically acceptable excipient. In exemplary embodiments, the genetically modified cells are NK cells that express CARs specific to Her2 and/or CD19 and are surface bonded with a plurality of nanoparticles (e.g., multilamellar liposomal vesicles) which encapsulate paclitaxel.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral or enteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art.

The pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Before administration to patients, formulants may be added to the engineered immune effector cell, or a population of cells containing a plurality of the engineered immune effector cell. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277.

### Kits

In various embodiments, the present invention provides kits comprising the pharmaceutical compositions described herein.

The kit is an assemblage of materials or components, including at least one of the inventive vectors and compositions. Thus, in some embodiments the kit contains a composition that has genetically modified cells expressing antigen-specific CARs and having bound on the cell surface a plurality of liposomes (for example, multilamellar liposomal vesicles) or other nanoparticles which encapsulate or otherwise carry one or more chemotherapeutic agents, as described above.

The exact nature of the components configured in the inventive kit depends on its intended purpose. In one embodiment, the kit is configured particularly for human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat, reduce the severity of, inhibit or prevent cancer in a subject. Optionally, the kit also contains other useful components, such as, measuring tools, diluents, buffers, pharmaceutically acceptable carriers, syringes or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of a composition. The packaging material generally has an external label which indicates the contents and/or purpose of the kit and/or its components.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention.

### Example 1

Experimental Methods

Cell Lines and Reagents:

MDA.MB.468 (ATCC HTB-132) and SKOV3 (ATCC HTB-77) tumor cell lines were maintained in a 5% CO2 environment in RPMI 1640 (Gibco) media supplemented with 10% FBS, 1% pen-strep, and 2 mM L-glutamine. NK92 cells (Dr. Jihane Khalife, Children's Hospital Los Angeles, ATCC CRL-2407) were maintained in MEM-α (Gibco) supplemented with 10% FBS, 10% horse serum, 1% NEAA, 1% pen-strep, 1% sodium pyruvate, 0.1 mM 2-β mercaptoethanol, 0.2 mM myo-inositol, and 2.5 μM folic acid. CD19⁺ SKOV3 (SKOV.CD19) cells were generated by transducing SKOV3 cells with lentivirus containing CD19 cDNA and sorting CD19⁺ cells with fluorescence-activated cell sorting (FACS).

PTX was purchased from Sigma-Aldrich (St. Louis, Mo.). All lipids were purchased from NOF Corporation (Japan): 1,2-dioleoyl-sn-glycero-3-phosphocholine (DOPC), 1,2-dioleoyl-sn-glycero-3-phospho-(10-rac-glycerol) (DOPG), and 1,2-dioleoyl-sn-glycero-3-phosphoethanolamine-N-[4-(p-maleimidophenyl)but-yramide (maleimide-headgroup lipid, MPB-PE).

Synthesis of Nanoparticles:

Liposomes were prepared based on the conventional dehydration-rehydration method (Joo, K, et al. (2013). Crosslinked multilamellar liposomes for controlled delivery of anticancer drugs. Biomaterials 34: 3098-3109; Moon, J, et al. (2011). Interbilayer-crosslinked multilamellar vesicles as synthetic vaccines for potent humoral and cellular immune responses. Nat Mater 10: 243-251). cMLVs were prepared from 1.5 μmol of lipids DOPC:DOPG:MPE-PE=40:10:50 mixed in chloroform and evaporated under argon gas before drying under a vacuum overnight to form dried lipid films. The lipid film was rehydrated in 10 mM Bis-Tris propane at pH 7.0. After the lipid was mixed through vigorous vortexing every 10 minutes for 1 hour, they underwent three cycles of 15-second sonication (Misonix Microson XL2000, Farmingdale, N.Y.) and rested on ice at 1-minute intervals after each cycle. A final concentration of 10 mM MgCl₂ was added to induce divalent-triggered vesicle fusion. The crosslinking of multilamellar vesicles (cMLVs) was performed by addition of Dithiothreitol (DTT, Sigma-Aldrich) at a final concentration of 1.5 mM for 1 h at 37° C. The cMLVs were collected by centrifugation at 14,000 g for 5 minutes and washed twice with PBS. The particles were suspended in filtered water, vortexed and sonicated prior to analysis. Morphological analysis of the multilamellar structure of vesicles was performed and confirmed by cryo-electron microscopy as previously studied by Joo, K., et al. The hydrodynamic size of cMLVs was measured by dynamic light scattering (Wyatt Technology, Santa Barbara, Calif.).

In Vitro Drug Encapsulation and Release:

The amount of incorporated paclitaxel in the cMLV(PTX) was determined by C-18 reverse-phase high-performance liquid chromatography (RPHPLC) (Beckman Coulter, Brea, Calif.). The cMLV(PTX) suspension was diluted by adding water and acetonitrile to a total volume of 0.5 mL. Extraction of paclitaxel was accomplished by adding 5 mL of tert-butyl methyl ether and vortex-mixing the sample for 1 min. The mixtures were centrifuged, and the organic layer was transferred into a glass tube and evaporated under argon. Buffer A (95% water, 5% acetonitrile) was used to rehydrate the glass tube. To test PTX concentration, 1 mL of the solution was injected into a C18 column, and the paclitaxel was detected at 227 nm (flow rate 1 mL/min). To obtain the release kinetics of PTX from cMLVs before and after cell conjugation, cMLV(PTX) and CAR.NK.cMLV(PTX) were incubated in 10% FBS-containing media at 37° C. and were spun down and resuspended with fresh media daily. The PTX was quantified from the removed media by HPLC every day.

Nanoparticle Conjugation with Cells and In Situ PEGylation:

Chemical conjugation of cMLVs to the cells was performed based on a method provided in previous studies (Huang, B, et al. (2015). Active targeting of chemotherapy to disseminated tumors using nanoparticle-carrying T cells. Sci Transl Med 77: 291ra294; Stephan, M, et al (2010). Therapeutic cell engineering with surface-conjugated synthetic nanoparticles. Nature Med 16: 1035-1041). We resuspended 10×10⁶ cells/mL in serum free MEM-α (Gibco) medium. Equal volumes of nanoparticles were resuspended in nuclease free water at different cMLV to NK cell conjugation ratios and incubated at 37° C. The cells and nanoparticles were mixed every 10 minutes for 30 minutes. After a PBS wash to remove unbound cMLVs from cells, cells were further incubated with 1 mg/ml thiol-terminated 2-kDa PEG at 37° C. for 30 minutes in media to quench residual maleimide groups on cell-bound particles. We performed two PBS washes to remove unbound PEG. For quantification of cell bound particles, particles were fluorescently labeled with the lipid-like fluorescent dye DiD (Invitrogen). Particle fluorescence was detected with flow cytometry and a fluorescent microplate reader. cMLVs were labeled with the lipid-like dye DiD and CAR.NK cells were stained with carboxyfluorescein diacetate succinimide ester (CF SE) (Invitrogen), which allowed the conjugation of cMLVs to NK cells to be easily detected using confocal microscopy.

Lentiviral and Retroviral Production and Transduction of NK92 Cells:

Our anti-Her2 CAR construct (Zhao, Y, et al. (2009). A Herceptin-based chimeric antigen receptor with modified signaling domains leads to enhanced survival of transduced T lymphocytes and antitumor activity. J Immunol 183: 5563-5574) was cloned into a lentiviral pCCW vector (a pCCL vector (Haas, D, et al (2003). The moloney murine leukemia virus repressor binding site represses expression in murine and human hematopoietic stem cells. J Virol 77: 9439-9450; Dull, T, et al (1998). A third-generation lentivirus vector with a conditional packaging system. J Virol 72: 8463-8471; Logan, A, et al (2004) Factors influencing the titer and infectivity of lentiviral vectors. Hum Gene Ther 15: 976-988)) with an additional WRE posttranscriptional regulatory region. The CAR consisted of the anti-Her2 scFv 4D5, a CD8 hinge and transmembrane region, and CD28, 4-1BB, and CD3ζ cytoplasmic regions. Our anti-CD19 CAR construct was cloned into a MP-71 retroviral vector backbone (Engels, B, et al. (2003) Retroviral vectors for high-level transgene expression in T lymphocytes. Hum Gene Ther 14: 1155-1168) and contained an anti-CD19 scFv, a CD8 hinge and transmembrane region, and CD28 and CD3ζ cytoplasmic regions. These plasmids were used to transfect HEK 293T cells in 30 mL plates using CaCl2 precipitation methods. Fresh media (high glucose DMEM supplemented with 10% FBS and 1% pen-strep) was plated onto the cells 4 hours after initial transfection. Supernatants were harvested and filtered (0.45 μm) 48 hours later. NK92 cells were transduced with fresh retrovirus. Lentiviral supernatant was concentrated (25,000 rpm for 90 minutes at 4° C.), resuspended in HBSS, and frozen at −80° C. until later use. NK92 cells were transduced with concentrated lentivirus at MOI 40; the titer was based on transduction of 293T cells.

CAR Detection on T Cell Surface:

Three days after transduction, anti-CD19 CAR.NK cells (1×10⁵) were incubated with biotinylated Protein L (Peprotech) at a volume ratio of 1:50 in PBS+4% FBS at 4° C. for 45 minutes and rinsed with PBS. The cells were subsequently incubated with streptavidin conjugated to FITC (Biolegend) at a volume ratio of 1:500 in PBS+4% FBS at 4° C. for 10 minutes, rinsed twice, and read using flow cytometry. Anti-Her2 CAR.NK cells (1×10⁵) were incubated with rhHer2-Fc chimera (Peprotech) at a volume ratio of 1:50 (2 μg/mL) in PBS at 4° C. for 30 minutes and rinsed with PBS. The cells were subsequently incubated with PE-labeled goat anti-human Fc (Jackson ImmunoResearch) at a volume ratio of 1:150 in PBS at 4° C. for 10 minutes, rinsed, and read using flow cytometry. Nontransduced NK cells served as a negative control.

Internalization Assay:

Quantification of cell cMLVs internalization was performed based on a method previously described (Huang, B, et al (2015). Active targeting of chemotherapy to disseminated tumors using nanoparticle-carrying T cells. Sci Transl Med 77: 291ra294; Stephan, M, et al (2010). Therapeutic cell engineering with surface-conjugated synthetic nanoparticles. Nature Med 16: 1035-1041). NK and CAR.NK cells were conjugated with 5 mole % 18:1 PE CF (1,2-dioleoyl-sn-glycero-3-phosphoethanolamine-N-(carboxyfluorescein) (ammonium salt) (Avanti, Polar Lipids)-tagged liposomes. After 2 PBS washes, cells were transferred to fibronectin (10ug/ml)-coated 96 well plates. After a 2 hour incubation time, half of the wells were treated with 100 μl trypan blue in HBSS (0.25 mg/mL), an extracellular fluorescence quenching dye, for 1 min in order to differentiate between membrane-bound and internalized liposomes. Trypan blue was removed by gentle vacuum aspiration and the cell uptake of liposomes was quantified by a fluorescence plate reader.

Cytokine Release Assay:

NK cells (1×10⁵ per well) were coincubated with target cells in 96-well plates at a 1:1 ratio for 6 hours at 37° C. 1 μg Brefeldin-A (Sigma) was added to each well to prevent protein transport. At the end of the incubation, cells were permeabilized using the CytoFix/CytoPerm kit (BD Biosciences) and stained for CD8 and IFN-γ using Pacific Blue-conjugated anti-human CD8 (Biolegend) and PE-conjugated anti-human IFN-γ (Biolegend). Unstimulated cells served as a negative control. Results were read using flow cytometry.

Cytotoxicity Assay:

Target cells (1×10⁴) were labeled with 5 μM carboxyfluorescein succinimidyl ester (CF SE, Life Technologies) as previously described (Han, X, et al. (2017). Masked chimeric antigen receptor for tumor-specific activation. Molecular Therapy 25: 274-284) and coincubated with NK cells at various ratios in 96-well plates for 24 hours at 37° C. The cells were then incubated in 7-AAD (Life Technologies) in PBS (1:1000 dilution) for 10 minutes at room temperature and analyzed via flow cytometry. Percentages of killed cells were calculated as [CFSE⁺7-AAD⁺ cells/(CFSE⁺7-AAD⁻+CFSE⁺7-AAD⁺)] cells, with live/dead gates based on control wells of target cells to account for spontaneous cell death.

NK92 and SKOV3 cells were seeded in 96-well plates at 2×10⁴ cells per well in 10% FBS-containing media and grown at 37° C. in the presence of 5% CO2 for 6 hours. Cells were incubated with various concentrations of cMLV (PTX) as previously described (Liu, Y, et al (2014). Codelivery of doxorubicin and paclitaxel by cross-linked multilamellar liposome enables synergistic antitumor activity. Mol Pharm 11: 1651-1661) and cell viability was assessed using the Cell Proliferation Kit II (XTT assay) from Roche Applied Science (Indianapolis, Ind.) according to the manufacturer's instructions. Cell viability percentage was determined by subtracting absorbance values obtained from media-only wells from the treated wells and then normalized by the control wells containing cells without drugs.

Transmigration Assay:

NK cell transmigration assays were performed in 24 mm diameter 3 μm pore size Transwell plates (Costar). NK cells either conjugated or unconjugated to cMLVs were plated on the upper wells and media was added to the lower wells. The chemoattractant CXCL9 (0.1 mg/ml, Peprotech) was added to the lower wells. After incubation at 37° C. for 6 hours, NK cells that had migrated into the lower chamber were counted.

In Vivo Biodistribution Study:

Female 6-10 weeks-old NOD.Cg-Prkdc^{scid}IL2Rγ^{tmlWjl}/SZ (NSG) mice were purchased from Jackson Laboratories (Bar Harbor, Me.). All mice were held under specific pathogen-reduced conditions in the animal facility of the University of Southern California (Los Angeles, Calif., USA). All experiments were performed in accordance with the guidelines set by the National Institute of Health and the University of Southern California on the Care and Use of Animals. A total of 3.5×10⁶ SKOV3.CD19 cells were inoculated subcutaneously into the flanks of NOD/scid/IL2rγ−/− (NSG) mice on Day −14, and tumors were allowed to grow until they reached 100 mm³. On Day 0, mice were injected intravenously through the tail vein with either cMLV(DiD) or CAR.NK.cMLV(DiD). 24, 48, and 72 hours after injection, mice were sacrificed and organs were analyzed for fluorescence intensity. DiD tissue fluorescence for each organ was quantified using the IVIS Spectrum imaging system and the percentage of injected dose per gram of tissue (% ID/g) was calculated.

Xenograft Tumor Model:

A total of 3.5×10⁶ SKOV3.CD19 cells were inoculated subcutaneously into the flanks of NSG mice on Day −14, and tumors were allowed to grow to 70-100 mm³. Mice were randomly divided into six groups of five mice each. On Days 0, 4, 7, and 11, the mice were injected intravenously through the tail vein with either PBS, cMLV(PTX) only, nontransduced NK cells only, CAR.NK cells only, mixed CAR.NK+cMLV(PTX) which were not conjugated together, or conjugated CAR.NK.cMLV(PTX). 5×10⁶ cells per mouse were injected each time in the groups that were given NK cells. Tumor growth and body weight of the mice were recorded until sacrifice. The tumor length and width were measured with a fine caliper, and tumor volume was calculated as ½×(length)×(width)².

Intratumoral PTX Concentration Measurements Ex Vivo:

Using high performance liquid chromatography (HPLC), the PTX concentration in the frozen tumor tissues was quantified as previously detailed (Liu, Y, et al (2014). Codelivery of chemotherapeutics via crosslinked multilamellar liposomal vesicles to overcome multidrug resistance in tumor. PLoS ONE 9: e110611). Briefly, thawed tumor tissues were chopped and homogenized in ethyl acetate, with a known concentration of docetaxel added to each sample as an internal standard. The samples were centrifuged and the organic layer was transferred to a clean tube. The organic layer was evaporated under a stream of argon and rehydrated in diluted acetonitrile. After running the samples on HPLC, the peak heights were analyzed to determine intratumoral PTX concentration.

Immunohistochemistry of Tumors, Cardiac Toxicity, and Confocal Imaging:

Tumors were excised, fixed, frozen, cryo-sectioned, and mounted onto glass slides. Frozen sections were fixed and rinsed with cold PBS. After blocking and permeabilization, the slides were washed with PBS and incubated with a terminal deoxynucleotidyl transferase dUTP nick end labeling (TUNEL) reaction mixture (Roche, Indianapolis, Ind.) for 1 hour and counterstained with 4′,6-diamidino-2-phenylindole (DAPI) (Invitrogen, Carlsbad, Calif.). Fluorescence images were acquired by a Yokogawa spinning-disk confocal scanner system (Solamere Technology Group, Salt Lake City, Utah) using a Nikon Eclipse Ti-E microscope. Illumination powers at 405, 491, 561, and 640 nm solid-state laser lines were provided by an AOTF (acousto-optical tunable filter)-controlled laser-merge system with 50 mW for each laser. All images were analyzed using Nikon NIS-Elements software. For quantifying TUNEL positive cells, four regions of interest (ROI) were randomly chosen per image at 10× magnification. Within one region, the area of TUNEL-positive nuclei and the area of nuclear staining were counted by Nikon NIS-Element software, with data expressed as % total nuclear area stained by TUNEL in the region. For cardiac toxicity, heart tissues were harvested 2 days after the last injection and were fixed in 4% formaldehyde. The tissues were frozen and then cut into sections and mounted onto glass slides. The frozen sections were stained with hematoxylin and eosin. Histopathologic specimens were examined by EVOS light microscopy.

Statistics:

The differences between two groups were determined with Student's t test. The differences among three or more groups were determined with a one-way analysis of variance (ANOVA).

### Example 2

### Anti-CD19 and Anti-Her2 CARs are Expressed in NK92 Cells

We confirmed the ability of NK92 cells to express anti-CD19 and anti-Her2 CARs, which consisted of an scFv-derived antigen binding domain, CD8 hinge and transmembrane region, CD28 and/or 4-1BB costimulatory domains, and CD3ζ signaling domain. Anti-CD19 CAR.NK cells were generated with retroviral transduction using the previously documented MP71 vector generously provided by Dr. Wolfgang Uckert. The anti-Her2 CAR.NK cells were generated with lentiviral transduction using a previously described trastuzumab-derived CAR in a pCCW vector, which is based off the pCCL vector⁴³⁻⁴⁵ with an added WRE posttranscriptional regulatory region. Transduced cells were sorted using fluorescence activated cell sorting to further increase the percentage of CAR⁺ cells (FIG. 1E). CAR expression was stable several months after initial transduction and sorting.

cMLVs are Stably Conjugated to the NK Cell Surface

Previous studies have shown that cross-linked multilamellar liposomal vesicles (cMLVs) were successfully incorporated with both hydrophobic and hydrophilic drugs. These vesicles were synthesized through covalently crosslinking functionalized headgroups of adjacent lipid bilayers using the conventional dehydration-rehydration method. Synthesized cMLV nanoparticles were stably conjugated to the reduced thiol groups present on the surface of NK cell via the thiol-reactive maleimide headgroups present on the lipid bilayer surface. High levels of free thiols were detected on the surfaces of lymphocytes. The conjugation was performed in two steps. First, NK cells and cMLVs were coincubated to induce particle coupling to free thiols on the cell surface. After the initial reaction, the cMLV-conjugated cells underwent in situ PEGylation to quench residual thiol reactive groups. To determine the maximum numbers of particles that could be conjugated per NK cell, we performed a serial dilution of the conjugation at different fluorescent-labeled cMLVs to cell ratios (2000:1, 1000:1, 500:1, 100:1, and 10:1). Between the conjugation ratio of 2000:1 and 1000:1, the number of conjugated liposomes per cell began to plateau and showed an average conjugation of approximately 150 nanoparticles per cell (FIG. 1B). From this data, we determined that the optimal ratio to use was 1000:1, as further increasing it did not increase the number of conjugated cMLVs on the cell surface. We used this ratio for all subsequent experiments. Confocal imaging was used to confirm and visualize the conjugation of cMLVs to the NK cell surface (FIG. 1C, FIG. 1F).

The major advantages of extended surface retention of nanoparticles on the surface of carrier cells are as follows: (1) prevention from immediate particle degradation due to internalization into degradative intracellular compartments and (2) sustained drug release from the particle-conjugated cells which allows for effective targeting of the drug to tumor cells. Nanoparticles can be endocytosed by a variety of cells, including endothelial cells and macrophages. However, for our study, it is crucial that the cMLVs remain on the NK cell surface. To address this, we performed an experiment to determine the internalization of these particles after conjugation. To determine whether these NK cells could also trigger liposome endocytosis, we conjugated NK cells with cMLVs tagged with a PE CF fluorescein dye, then warmed the cells to 37° C. and assessed cell-associated fluorescence over time. Attachment of cMLVs to NK cells did not trigger cell uptake of these particles and particles bound to NK cells remained at the cell surface as shown in FIG. 1D.

CAR.NK Cells have Greater Cytotoxic Effects Against Antigen-Expressing Target Cells In Vitro and are Less Sensitive to PTX

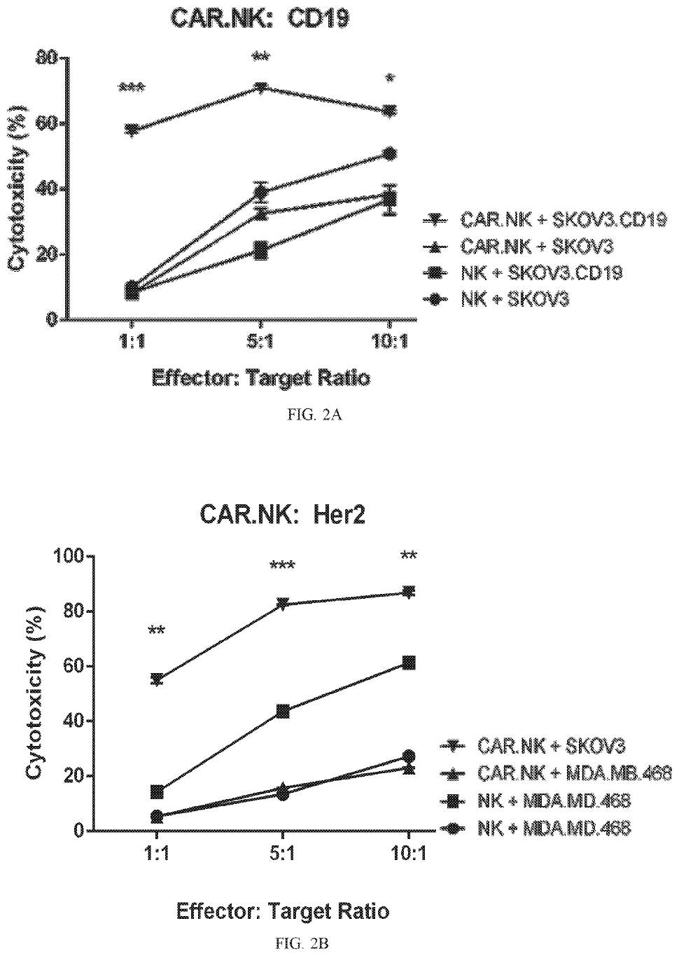

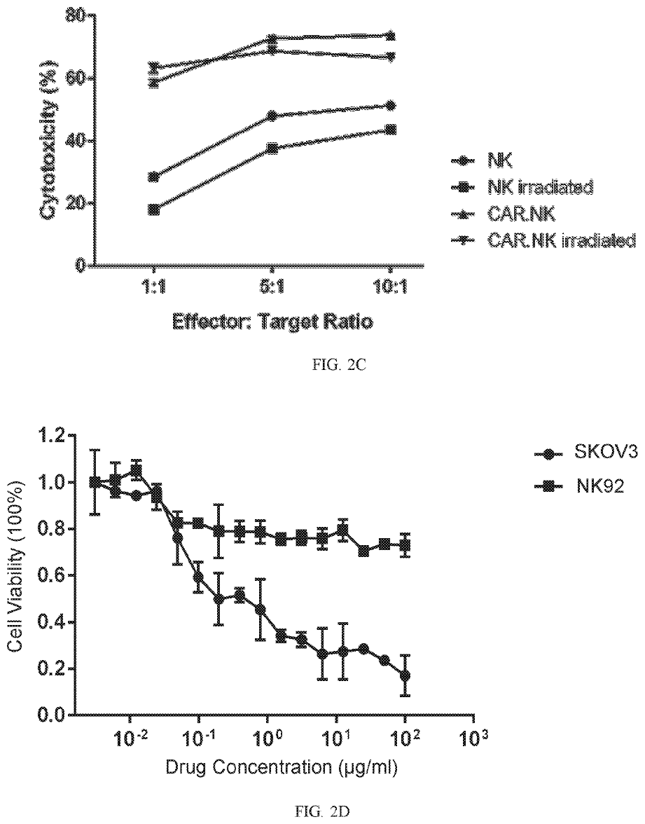

We assessed the ability of CAR.NK cells to trigger cytotoxic effects against the appropriate antigen-expressing target cells by coincubating nontransduced NK or CAR.NK cells with various target cell lines and reading the results with flow cytometry. We used lentivirus to transduce SKOV3 cells to express CD19 (SKOV.CD19) to serve as target cells for our anti-CD19 CAR.NK cells. Both CD19 and Her2-targeting CAR.NK cells demonstrated significantly greater cytotoxicity against the antigen-expressing target cells (SKOV.CD19 and SKOV3, respectively) compared with either nontransduced NK cells or CAR.NK cells coincubated with target cells that did not express the cognate antigen (SKOV3 and MDA.MB.468, respectively, FIG. 2A, FIG. 2B). These trends were observed at all effector-to-target ratios (p<0.01 for 1:1 and 10:1, p<0.001 for 5:1) and indicated CAR-mediated cell killing.

Since NK92 cells originate from a patient with NK cell lymphoma, these allogenic cells are irradiated prior to clinical use to prevent them from proliferating in vivo. Irradiation did not affect the cytotoxic capabilities of our CAR.NK cells (FIG. 2C). We also performed a cell viability assay to demonstrate that SKOV3 cells were more sensitive to PTX than NK cells were (FIG. 2D). This ensures that the NK cells can carry enough PTX to kill target cells without succumbing to PTX-induced toxicity themselves. cMLVs conjugated to NK cells also release the majority of their PTX payload by Day 3 (FIG. 2E).

CAR.NK Function is Unaffected by cMLV Conjugation and Enhanced with cMLV(PTX) Conjugation In Vitro

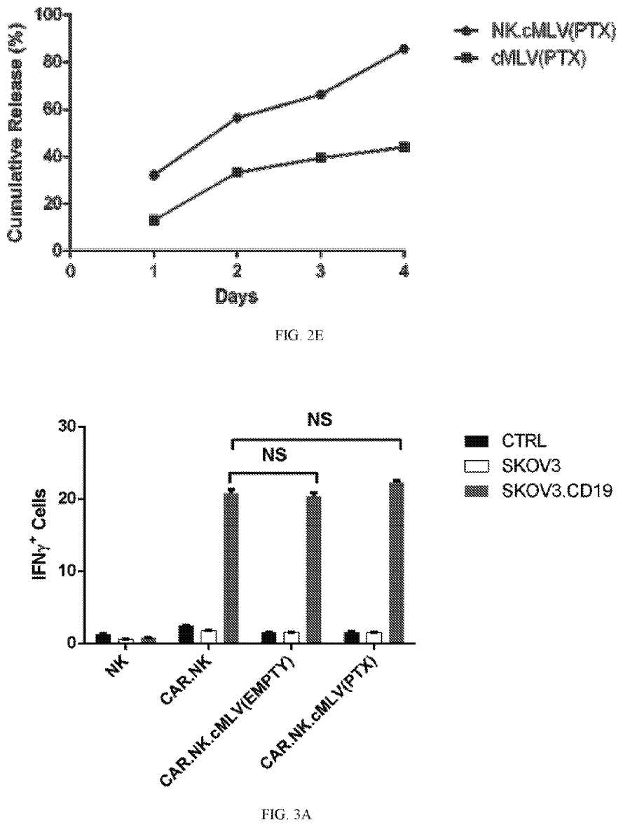

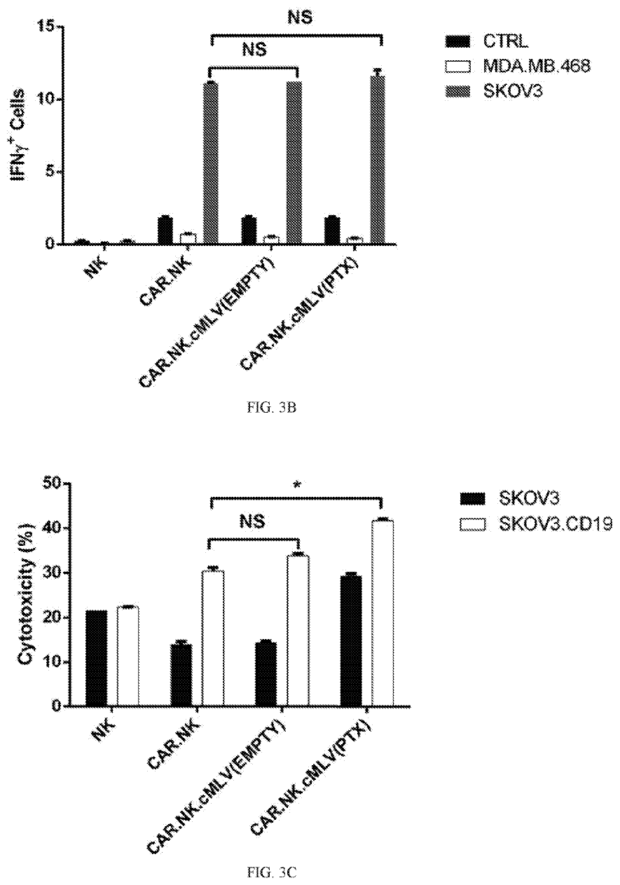

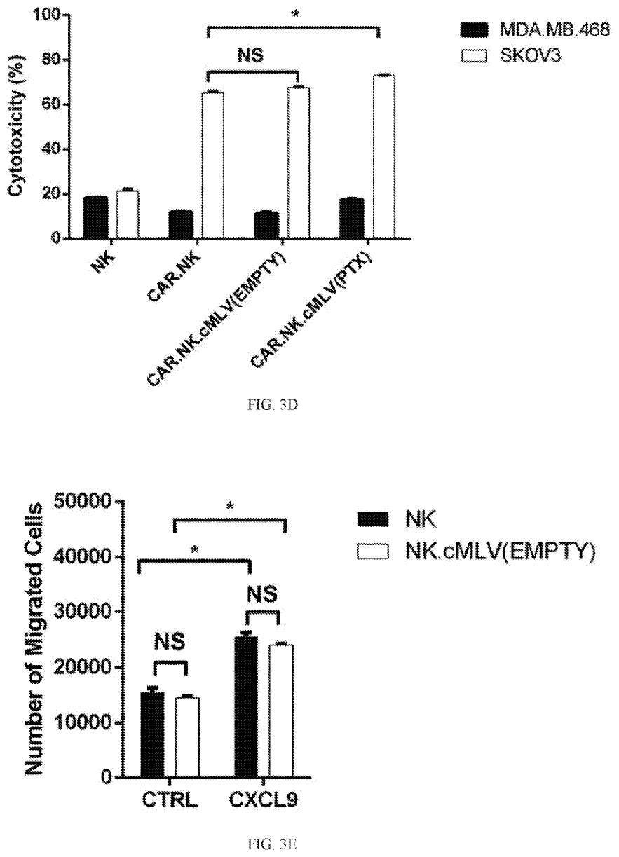

We ensured that the conjugation of cMLVs to the CAR.NK cell surface does not affect the functionality of the CAR.NK cell itself. To detect NK cell activation upon antigen binding, we performed an IFN-γ release assay, coculturing various target cell lines with NK cells with or without cMLV conjugation. None of the CAR.NK cells reacted when incubated alone or when cocultured with target cells without the cognate antigen, but coincubation with the correct antigen-expressing target cells resulted in significantly greater percentages of IFN-γ⁺ cells from both anti-CD19 and anti-Her2 CAR.NK cell lines (p<0.05) demonstrating specificity towards the appropriate TAA. When the CAR.NK cells were conjugated to either empty cMLVs containing no drug (CAR.NK.cMLV(EMPTY)) or PTX-loaded cMLVs (CAR.NK.cMLV(PTX)), IFN-γ release was not significantly different from that of unconjugated CAR.NK cells (FIG. 3A, FIG. 3B).

We repeated the cytotoxicity assays using an effector-to-target ratio of 1:1 with CAR.NK cells that were unconjugated, conjugated to empty cMLVs (CAR.NK.cMLV(EMPTY)), or conjugated to PTX-loaded cMLVs (CAR.NK.cMLV(PTX)). CAR.NK.cMLV(EMPTY) did not have significantly affected cell killing, but cytotoxicity against target cells was significantly increased with CAR.NK.cMLV(PTX) (FIG. 3C, FIG. 3D). These data indicate that while empty cMLVs do not affect CAR.NK function, the release of PTX from cMLVs in proximity to the target cells further boosted cytotoxic effects.

Finally, we monitored NK migration with or without cMLV conjugation. In order to affect an antitumor response, NK cells must extravasate into and migrate within the tumor site in response to chemoattractants. To ensure that cMLV conjugation to the NK surface did not impact cell migration, we performed NK cell transmigration assays. The chemoattractant CXCL9 was used to promote NK cell migration to the lower chamber of the wells. There were significantly more migrated NK cells in the lower chamber when CXCL9 was used as an attractant compared to the plain media control (p<0.05), but there was no significant difference between conjugated and unconjugated groups, indicating that conjugation of cMLVs to the cell surface did not impact NK migratory abilities (FIG. 3E).

CAR.NK.cMLV Enhances Delivery of cMLVs to the Tumor Site

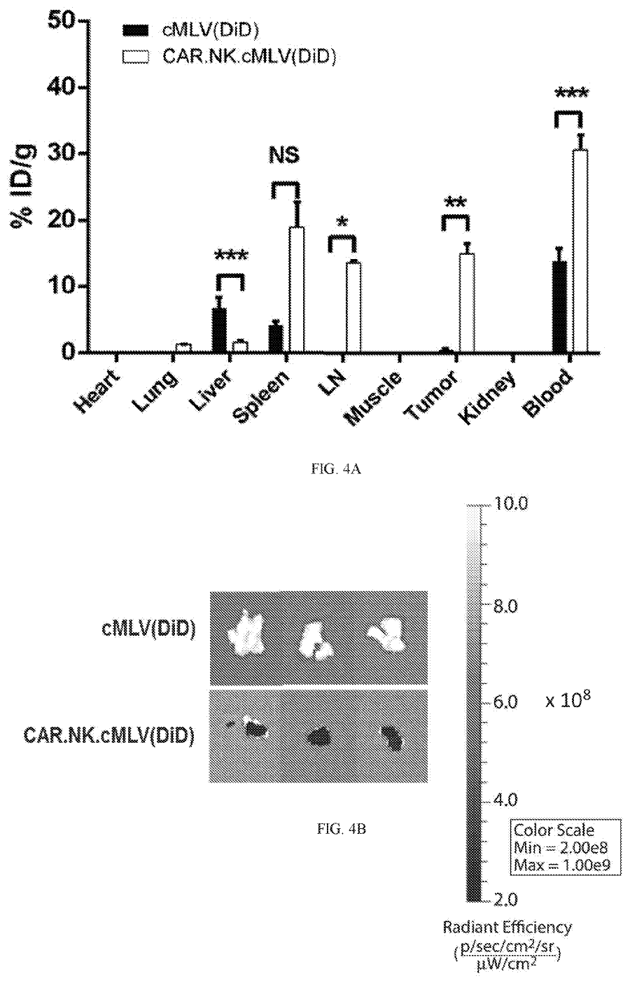

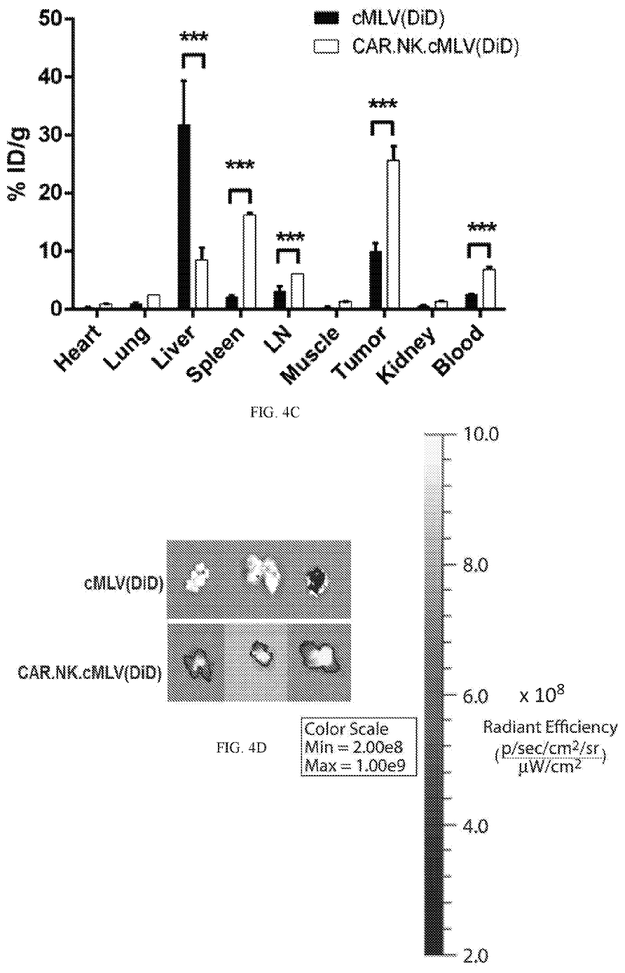

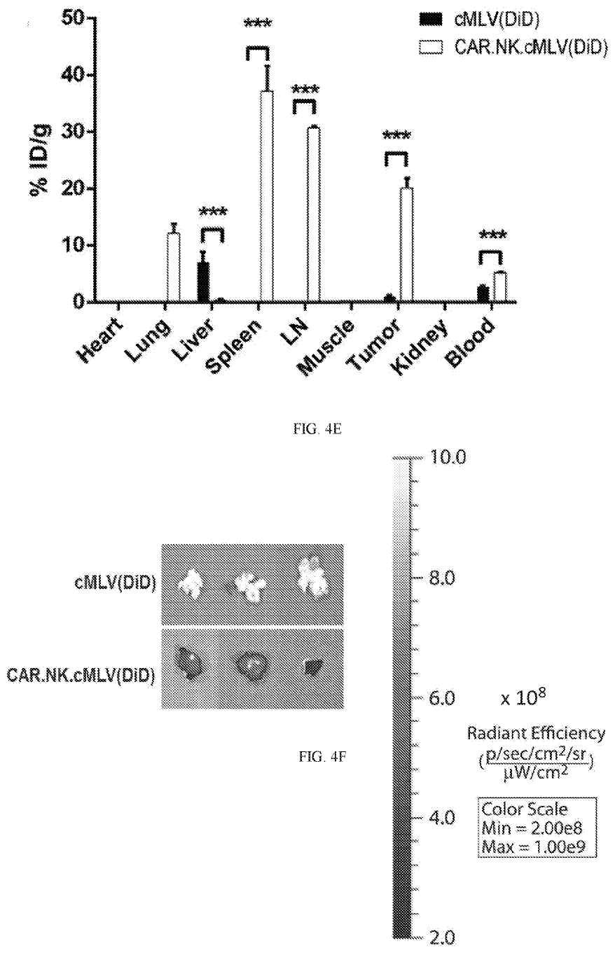

After confirming the functionality of our cMLV(PTX)-conjugated CAR.NK cells in vitro, we performed a biodistribution study to determine if CAR.NK cells enhanced cMLV homing to the tumor site. The fluorescent dye DiD was used to tag cMLVs (cMLV(DiD)) and track their presence in various organs. NSG mice were subcutaneously injected with SKOV.CD19 cells. Two weeks after tumor inoculation, mice were intravenously injected with cMLV(DiD) or conjugated CAR.NK.cMLV(DiD). Mice were sacrificed and organs were analyzed for fluorescence signal at various time points. At 24 hours (FIG. 4A, FIG. 4B), most of the cMLVs from both groups were still circulating in the blood. The CAR.NK.cMLV(DiD) group had significantly more cMLVs in the blood (p<0.001), lymph node (p<0.05), and tumor (p<0.01), while the cMLV(DiD) group had significantly more accumulation in the liver (p<0.001). At 48 hours (FIG. 4C, FIG. 4D), most of the cMLV(DiD) group had accumulated in the liver, but the CAR.NK.cMLV(DiD) group had significantly more cMLVs in the blood, lymph node, spleen, and tumor (p<0.001). By 72 hours (FIG. 4E, FIG. 4F), most of the cMLV(DiD) signal was gone, with only small amounts detectable in the liver, blood, and tumor. In contrast, the CAR.NK.cMLV(DiD) group had significantly more cMLVs in the blood, lymph node, spleen, and tumor (p<0.001). Overall, cMLV(DiD) without a cell acting as a chaperone were likely cleared by the liver, as hepatic clearance serves as the main clearance route for particles too large to be cleared by the kidneys. In contrast, cMLV(DiD) that were conjugated to CAR.NK cells were able to home to the tumor site.

CAR.NK.cMLV(PTX) Enhances Antitumor Efficacy In Vivo

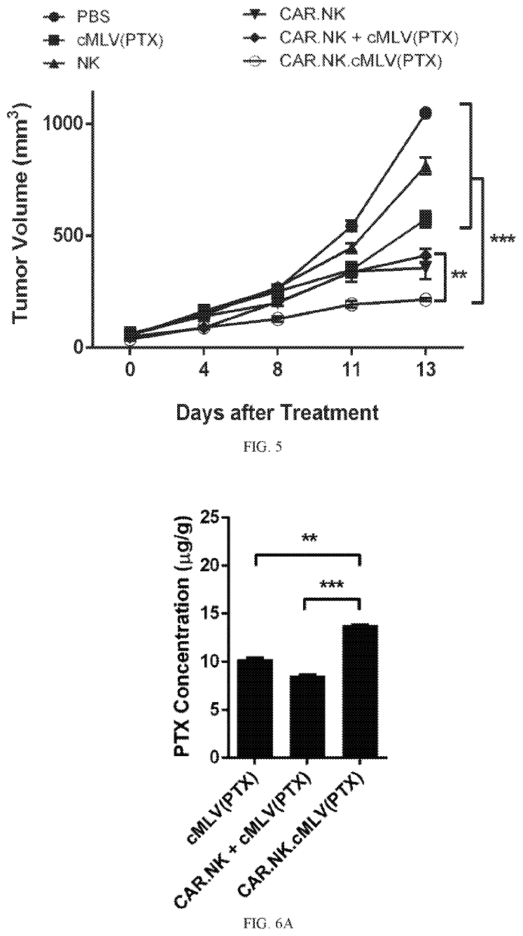

We established a mouse xenograft model to observe the effects of the anti-CD19 CAR.NK cells in vivo. NSG mice were subcutaneously injected with SKOV.CD19 cells. Two weeks after tumor inoculation, mice were randomly divided into six groups and injected via tail veins (intravenously) with (1) PBS as a control, (2) cMLV(PTX) only, without any cellular component, (3) nontransduced NK cells only, (4) CAR.NK cells only, (5) mixed cMLV(PTX)+CAR.NK which were coinjected but not conjugated, and (6) conjugated CAR.NK.cMLV(PTX) cells. In group (6), 0.1 mg PTX was injected per mouse. For a 20 g mouse, 5 million cells were administered per injection, for a total of four injections. Mice treated with CAR.NK.cMLV(PTX) had significantly slowed tumor growth compared to PBS, cMLV(PTX), and NK groups (p<0.001), and significantly slowed tumor growth compared to CAR.NK and CAR.NK+cMLV(PTX) groups as well (p<0.01, FIG. 5). These data support the hypothesis that both immunotherapeutic effects from the NK cells and chemotherapeutic effects from the PTX play a role in the killing of tumor cells, as either component alone was not as effective as when the two were combined. Furthermore, even the mice treated with CAR.NK+cMLV(PTX) did not have as great an antitumor response as did the mice treated with CAR.NK.cMLV(PTX). This demonstrates that the conjugation between the drug and the NK cell is crucial to receiving the full benefits of the treatment system, and that the CAR.NK cells are facilitating the delivery of PTX to the tumor site for enhanced anticancer effects.

CAR.NK.cMLV(PTX) Enhances PTX Delivery into Tumor Site

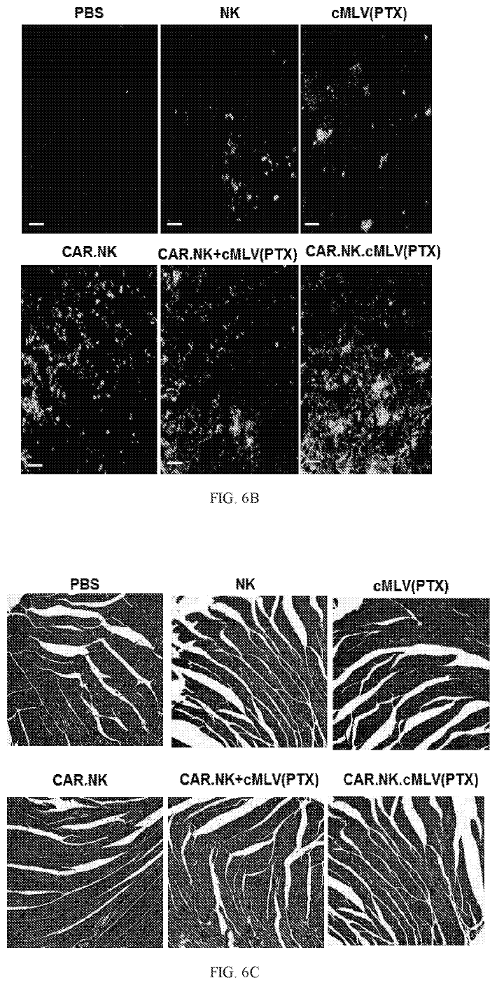

We performed ex vivo analysis of our mouse xenograft tumor model to support our hypothesis that CAR.NK cells facilitate PTX delivery into the tumor site. Using high performance liquid chromatography (HPLC), we quantified the intratumoral PTX concentrations in mice treated with PTX, including the groups cMLV(PTX), CAR.NK+cMLV(PTX), and CAR.NK.cMLV(PTX). The conjugated group, CAR.NK.cMLV(PTX), had significantly higher PTX concentrations within the tumor tissue compared to the cMLV(PTX) or CAR.NK+cMLV(PTX) (p<0.01 and p<0.001, respectively, FIG. 6A).

We also used confocal imaging to visualize apoptotic cells in tumor tissues fixed on glass slides. There were more terminal deoxynucleotidyl transferase dUTP nick end labeling (TUNEL)⁺ cells in groups treated with CAR.NK cells than in control or NK cell groups, indicating greater cell killing from CAR.NK cells, as shown in FIG. 6B. A higher level of cell apoptosis was observed in the group treated with CAR.NK+cMLV(PTX) when compared to cMLV(PTX) treatment only, but the degree of cell apoptosis was similar when compared to CAR.NK treatment. Notably, CAR.NK.cMLV(PTX)-treated tumors had the greatest degree of cell apoptosis, indicating synergistic efficacy induced by the co-localized delivery of the drug and CAR.NK cells.

Finally, as the therapeutic effect of PTX is limited by its cardiotoxicity, slices of fixed heart tissue stained with hematoxylin and eosin were imaged with light microscopy. Cardiotoxicity was defined as myofibrillary loss and disarray, as well as cytoplasmic vacuolization. We observed no damage to the cardiac tissues in any of the treatment groups (FIG. 6C). Since our delivery was targeted, we were able to use a very low dose of PTX (0.5 mg/kg) compared to those used in conventional PTX-based treatments⁵⁷⁻⁵⁹, thus resulting in minimal cardiotoxicity.

Our system combines nanoparticle-based drug delivery with immunotherapy to produce a cell-mediated, active targeting strategy. In vitro, we demonstrate that cMLV conjugation to NK cells does not trigger endocytosis, even though NK cells have phagocytotic capabilities. The particles remain on the NK cell surface, perhaps in part due to the size of the cMLVs—previous studies have shown that surface-conjugated particles larger than 50 nm in diameter are not efficiently internalized. Furthermore, the covalent linkage of maleimide-functionalized cMLVs to free thiols on immune cell surfaces has been shown to be stable for days after initial conjugation and even after cell division. While the exact mechanisms of this prolonged surface retention remain to be discovered, the maleimide-thiol conjugation strategy has been shown to be a promising method of immune cell surface engineering.

We also have demonstrated in vitro that CAR.NK cells can specifically kill antigen-expressing cancer cells, that cMLV conjugation does not adversely affect NK cell function, and that conjugation of cMLV(PTX) to CAR.NK cells further augments cytotoxicity. While many studies of CAR.NK cells include results from cytotoxicity assays but not from cytokine release assays, we show that CAR.NK cells release IFN-γ in response to TAA⁺ target cells. Neither CAR.NK cells coincubated with TAA⁻ target cells nor nontransduced NK cells coincubated with any target cells release IFN-γ. These results indicate that the enhanced cytotoxicity of CAR.NK cells was accompanied by an increase in IFN-γ release. In addition to sensitizing tumor cells to NK cytotoxicity, IFN-γ release by both primary NK cells and NK cell lines signals to surrounding immune cells, including T cells, dendritic cells, monocytes, and macrophages, initiating broader adaptive and innate immune responses.

Our in vivo biodistribution study further supports that CAR.NK cells enhance nanoparticle accumulation within the tumor site. Mice treated with cMLV(DiD) without a cell chaperone had significantly greater cMLV accumulation in the liver, likely indicating hepatic clearance as commonly observed with larger liposomes. However, the CAR.NK.cMLV(DiD)-treated mice had significantly greater cMLV accumulation at the tumor site. Additionally, significantly higher signal was observed in organs to which NK cells naturally home, such as the spleen and lymph nodes. Our in vivo and ex vivo data provide evidence that CAR.NK cells facilitate the delivery of the chemotherapeutic drug PTX to the tumor site, slowing tumor growth and increasing intratumoral PTX concentrations more effectively than any other treatment group, including coadministered but not conjugated CAR.NK and cMLV(PTX). Finally, we were able to use a low dose of PTX and did not observe any cardiotoxicity.

We found that certain doses of PTX can kill tumor cells but not NK92 cells, creating a therapeutic window in which we can use NK92 cells to deliver this chemotherapeutic drug to kill tumor cells but not the carrier cells. However, we do not believe that this system is limited to PTX delivery. For example, murine T cells have been shown to deliver the anticancer drug SN-38 to lymphoma sites in vivo using drug-loaded nanocapsules conjugated to the cell surface. SN-38 effectively killed lymphoma cells but was not toxic to the T cell carriers. Another study demonstrated that primary human T cells can enhance antitumor immune responses using surface-conjugated liposomes carrying the proinflammatory cytokines IL-15 and IL-21. Surface engineering of immune cells has allowed a number of drugs or adjuvants to be delivered to the tumor site. To our knowledge, we present the first study of surface-engineered NK cells as well as the first study using CAR.NK cells for tumor-targeted drug delivery. We believe that our CAR.NK-mediated drug delivery system can be expanded to include not only the delivery of traditional chemotherapeutic agents, but other anticancer treatments such as immunomodulators and small molecules that affect the tumor microenvironment.

Cancer immunotherapy has attracted much attention as an alternative or addition to chemotherapy, and currently a few clinical trials are using CAR-engineered T (CAR-T) cells to target patients with relapsed solid cancers, such as pancreatic, ovarian, prostate, and lung cancers. However, CAR-T therapy relies on the ex vivo expansion of the patient's autologous T cells, which presents logistical issues and delays the start of the treatment while cells are in preparation (typically 2-3 weeks for the expansion of CAR-engineered immune cells for clinical use). These issues could be ameliorated in part by using an allogenic cell line instead of autologous cells; while there are few functional cytotoxic T cell lines available, there are several functional, immortal NK cell lines. Of these NK cell lines, NK92 is the most promising and the only NK cell line used in clinical trials.

There are a number of potential benefits to using CAR-engineered NK92 cells over CAR-T cells. CAR-engineered NK92 cells may provide an alternative “off-the-shelf” vehicle for CAR-based therapy as well as provide more targeted drug delivery to the tumor site through surface engineering. NK92 cells double every 2-4 days, allowing for easy expansion, modification, and storage under good manufacturing practice (GMP) conditions. NK92 cells are identical to the parental cell line, eliminating problems with donor variability. There would be no lag time required for the ex vivo expansion and modification of autologous immune cells, which is especially crucial in patients with aggressive cancers, where a treatment delay of days to weeks could impact outcome. NK92 cells are safe to use clinically if irradiated, which prevents proliferation. This decreases the risk of off-target effects compared to CAR-T cells. Short-lived CAR-engineered NK92 cells can be treated as a “living drug”, redosing as necessary. Finally, allogenic NK92 cell-based therapies are less expensive than autologous CAR-T cell therapies—one group estimated that each CAR-T protocol costs upwards of $250,000 per patient, but NK92 cells used in the clinic cost around $20,000 per patient.

We have demonstrated that CAR.NK cells conjugated to PTX-loaded cMLVs offer targeted drug delivery and improved antitumor efficacy. We believe that targeted drug delivery using surface-engineered CAR.NK cells is widely applicable, as both the CAR target and the drug payload potentially can be altered to treat a variety of cancer types. Overall, this study shows a promising combination of immunotherapy and drug delivery for enhanced antitumor treatment.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. An engineered immune effector cell, comprising:
a natural killer (NK) cell; and
a plurality of nanoparticles bound to the surface of the NK cell;
wherein the NK cell contains polynucleotides encoding one or more chimeric antigen receptors (CARs), the CAR comprising an extracellular antigen specific domain, and
wherein the plurality of nanoparticles encapsulates a chemotherapeutic agent in an amount that causes no or less than 15% cytotoxicity to a population of NK cells.

2. A cell population, comprising a plurality of the engineered immune effector cell of claim 1,
wherein each engineered immune effector cell comprises:
a. a natural killer (NK) cell, further comprising polynucleotides encoding one or more chimeric antigen receptors (CARs), the CAR comprising an extracellular antigen specific domain; and
b. a plurality of nanoparticles bound to the surface of the NK cell, at least one nanoparticle encapsulating a chemotherapeutic agent;
and wherein the cell population has a total amount of the chemotherapeutic agent causing no or less than 15% cytotoxicity to the plurality of engineered immune effector cells, yet effective for inhibition or killing of tumor cells.

3. The engineered immune effector cell of claim 1, wherein the nanoparticles are liposomes comprising crosslinked multilamellar liposomal vesicles (cMLVs).

4. The engineered immune effector cell of claim 1, wherein the CAR binds to CD19.

5. The engineered immune effector cell of claim 1, wherein the CAR binds Her2.

6. The engineered immune effector cell of claim 1, wherein the CAR is a bispecific CAR and binds CD19 and Her2.

7. The engineered immune effector cell of claim 1, wherein the nanoparticles are cMLVs and the chemotherapeutic agent is paclitaxel.

8. The engineered immune effector cell of claim 1, wherein the nanoparticles are cMLVs and the cMLVs are chemically bonded to the surface of the NK cell at a number ratio between 100:1 and 150:1.

9. A pharmaceutical composition comprising the engineered immune effector cell of claim 1 and a pharmaceutically acceptable carrier.

10. A pharmaceutical composition comprising the cell population of claim 2 and a pharmaceutically acceptable carrier.

11. A method for treating a subject with a cancer comprising administering to the subject an effective amount of the engineered immune effector cell of claim 1.

12. A method for treating a subject with cancer comprising administering to the subject a therapeutically effective amount of the pharmaceutical composition of claim 10.

13. The method of claim 12, wherein the pharmaceutical composition is administered via intravenous infusion.

14. The method of claim 12, wherein the subject has undergone total body irradiation (TBI), IgG1 antibody administration, or both, prior to the administration of the pharmaceutical composition.

15. The method of claim 12, further comprising one or more subsequent administrations of the pharmaceutical composition at weekly, biweekly, triweekly, monthly, quarterly or yearly intervals.

16. The method of claim 12, wherein the subject has one or more cancers selected from the group consisting of leukemia, melanoma, renal cancer, prostate cancer, breast cancer, colon cancer, and lung cancer,
